(19) United States
(12) Patent Application Publication
(10) Pub. No.: US 2024/0043791 A1
(43) Pub. Date: Feb. 8, 2024

(54) MODIFIED BACTERIUM FOR OXALATE DEGRADATION AND USES THEREOF

(71) Applicant: Massachusetts Institute of Technology, Cambridge, MA (US)

(72) Inventors: Avihu YONA, Cambridge, MA (US); Jeffrey GORE, Cambridge, MA (US)

(21) Appl. No.: 18/258,648

(22) PCT Filed: Dec. 22, 2021
(86) PCT No.: PCT/US2021/064855

§ 371 Date: Jun. 21, 2023

Related U.S. Application Data
(60) Provisional application No. 63/129,776, filed on Dec. 23, 2020.
(60) Provisional application No. 63/129,790, filed on Dec. 23, 2020.

(51) Int. Cl. C12N 1/20; A61P 3/00; C12N 1/38

(52) U.S. Cl. CPC C12N 1/205; A61P 3/00; C12N 1/38; C12R 2001/19

(57) ABSTRACT

Modified bacterium capable of degrading oxalate, mutant proteins that facilitate oxalate degradation, and methods for their use are disclosed.

### CROSS-REFERENCE

This application claims priority to U.S. Provisional Patent Application Ser. Nos. 63/129,776 filed Dec. 23, 2020, and 63/129,790 filed Dec. 23, 2020 each incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Dec. 21, 2021 having the file name “21-1553-WO-SeqList_ST25.txt” and is 67 kb in size.

### BACKGROUND

Gut microbe metabolism is implicated in the etiology and progression of several human health conditions, including cancers and inflammatory, autoimmune, and metabolic disorders. As a result, “functional probiotics” with potentially useful metabolic features are a promising new avenue for therapeutics. However, the human gut microbiome is a complex multispecies community where host-microbe and interspecies interactions determine which microbes can be metabolically active. Because exogenous microbes can be maladapted to the gut environment, microbes with potentially therapeutic metabolic pathways in vitro are often inactive once introduced to the gut.

Oxalate is ubiquitous in plants and plant-derived foods consumed by humans and other mammals. Urinary oxalate excretion at concentrations exceeding 40-45 mg per 24 hours is hyperoxaluria, which can cause significant morbidity and mortality, including the development of renal stones (kidney stones), nephrocalcinosis (increased calcium in the kidney) and most significantly, End Stage Renal Disease (ESRD). Currently available treatments for hyperoxalurias are inadequate

### SUMMARY

In one aspect, the disclosure provides modified a bacterium that comprises one or more mutations compared to a corresponding wild-type bacterium, wherein the modified bacterium can degrade oxalate and the corresponding wild-type bacterium cannot degrade oxalate. In one embodiment, the one or more mutations comprise mutations in one or more genes selected from the group consisting of eutE, eutN, adhE, icd, sthA, pka, aceK, nadR, iclR, rpoB, mg, tosA, and rpoS, all based on naming convention for E. coli; or homologues or orthologues thereof. In another embodiment, the one or more mutations comprise mutations in one or more genes selected from the group consisting of eutN, adhE, sthA, and nadR. In a further embodiment, the one or more mutations comprise mutations in one or more genes selected from the group consisting of eutN, adhE, and eutE. In another embodiment, the bacterium is a member of a genus selected from the group consisting of Bacteroides, Bifidobacterium, Clostridium, Escherichia, Streptococcus, Lactobacillus and Lactococcus. In a further embodiment, the bacterium is of the genus Escherichia. In another embodiment the disclosure provides a bacterial populations, comprising a plurality of the modified bacterium of any embodiment or combination of embodiments of the disclosure.

In another aspect, the disclosure provides an isolated protein, comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of one of the following:
(a) SEQ ID NO: 1, and including 1, 2, 3, 4, or all 5 residues selected from 77H, 546D, 581C, 600D, and/or F837L;
(b) SEQ ID NO: 1, and including 2, 3, 4, 5, or all 6 residues selected from 77H, 546D, 581C, F583L, 600D, and/or F837L;
(c) SEQ ID NO: 2, and including one or both residues selected from 10L and 14Q;
(d) SEQ ID NO: 3, and including one or more residues selected from 58L, 60V, and 63T;
(e) SEQ ID NO: 4, and including residue 403L;
(f) SEQ ID NO: 5 and including residue 150T and/or A268T;
(g) SEQ ID NO: 6 and including residue D158G;
(h) SEQ ID NO:7 and including residue R565C;
(i) SEQ ID NO:8 and including residue Q137*, wherein the asterisk represents a truncation of the protein at position 137;
(j) SEQ ID NO:9 and including residue A532E;
(k) SEQ ID NO:10 and including residue G66V;
(l) SEQ ID NO:11 and including residue R394P;
(m) SEQ ID NO:12 and including residue Y919C; and
(n) SEQ ID NO:13 and including residue G596W.

In other embodiments, the disclosure provides nucleic acids encoding the polypeptide of the disclosure; expression vectors comprising the nucleic acid of the disclosure operatively linked to a regulatory sequence; host cells comprising the polypeptide, nucleic acid, and or expression vector of any embodiment of the disclosure; and compositions comprising the modified bacterium, bacterial population, polypeptide, nucleic acid, expression vector, and/or host cell of any embodiment of the disclosure.

In another aspect, the disclosure provides methods for reducing oxalate, methods for treating or limiting development of a disorder in which oxalate is detrimental in a subject, methods for treating or limiting development of hyperoxaluria, and methods for treating or limiting development of kidney stones, comprising administering to a subject in need thereof the modified bacterium, bacterial population, polypeptide, nucleic acid, expression vector, host cell, and/or composition of any embodiment of the disclosure.

### DESCRIPTION OF THE FIGURES

FIG. 1(A). E. coli isolated from different individuals are highly diverse—the fraction of proteins with higher than 95% amino acid similarity ranges between 60-70%, except between strains isolated from the same individual (Ec2, Ec3).

FIG. 1 (B) Lab evolution scheme to select for de novo metabolism of oxalate in gut isolates—in a microaerobic chamber, populations are evolved by serial dilution on minimal medium with glycerol and oxalate (M9+0.05% glycerol and 0.2% oxalate). When samples of evolving populations form colonies on plates with oxalate as a sole source of carbon and energy, glycerol is omitted from the evolution medium.

FIG. 2(A). De novo metabolism of oxalate as a sole source of carbon and energy in microaerobic conditions—Final yields of evolved populations after dilution 60, measured on M9+oxalate 0.2%. Error bars represent s.d. of OD measurements of three biological replicates. Before evolution, none of the isolates could utilize oxalate. After evolution, all populations grow robustly on oxalate as a sole source of carbon and energy in microaerobic but not aerobic conditions.

FIG. 2(B). Whole genome sequencing reveals three genes with repeated mutations across isolates—repeated mutations in eutE, eutN and adhE are shown (light) as well as mutations that occurred in genes that mutated only in a single population (dark). For details on promoter mutations in AdhE see FIG. 4.

FIG. 2(C). Repeated mutations suggest an evolved interaction between oxalate and a dehydrogenase—out of 33 total mutations, 15 mutations were identified in EutE (a microcompartment-enclosed aldehyde dehydrogenase), EutN (a selectively permeable component of the microcompartment shell enclosing EutE) and AdhE (a cytosolic alcohol-aldehyde dehydrogenase). These mutations suggest that ancestral strains lacked dehydrogenase activity essential for oxalate utilization. EutE signal-sequence mutations relocate EutE to the cytosol, where it is contact with oxalate and oxalyl-CoA. Similarly, EutN mutations might allow dehydrogenase activity toward oxalate metabolites by EutE inside the microcompartment. AdhE mutations near the alcohol dehydrogenase active site might increase activity toward non-native substrates

FIG. 2(D) List of oxalate-metabolizing single colonies isolated from the four evolving populations and fully sequenced to reveal mutations/mutation-combinations that confer the ability to utilize oxalate.

FIG. 3(A). Mutations in EutE occurred in its microcompartment signal sequence (SEQ ID NO: 14).

FIG. 3(B). Mutations in EutE disrupt its microcompartment signal sequence—Annotated signal sequence alignment of native ssPduD, ssPduP and ssEutE with the tag variants ssEutE(V10L) and ssEutE(L14Q). The common signal sequence motif identified by Jakobson and colleagues is indicated by residues highlighted (SEQ ID NOs 15-19, from top to bottom).

FIG. 3(C). Mutations in the microcompartment shell protein EutN are pore-facing—All mutations observed in EutN are localized around the pore. Mutated residues are shown as an overlay on the crystal structure of EutN (PDB 2Z9H) with the wildtype residues shown as a mesh contour.

FIG. 4(A). Mutations in AdhE cluster in the alcohol dehydrogenase domain along the interface with the aldehyde dehydrogenase Two mutations occurred near the alcohol dehydrogenase active site (G600D and G546D). Mutated residues are shown on the crystal structure of AdhE (PDB 6AHC).

FIG. 4(B). Mutations in the 5′-UTR of AdhE strengthen −10 promoter element—Three mutations occurred in the promoter of AdhE, all in the evolved EcN population. Promoter analysis software predicts that one mutation strengthens the −10 element, while a second strengthens the cAMP receptor protein (CRP) binding site (SEQ ID NO: 20).

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

As used herein, “about” means +/−5% of the recited value.

In a first aspect, the disclosure provides modified bacterium that comprises one or more mutations compared to a corresponding wild-type bacterium, wherein the modified bacterium can degrade oxalate and the corresponding wild-type bacterium cannot degrade oxalate. As used herein, “degrade oxalate” refers to any reduction or degradation of oxalate when compared to an appropriate control. Oxalate degrading activity includes formyl-CoA/oxalyl-CoA transferase activity, oxalyl-CoA decarboxylase activity, or any activity employed in an enzymatic pathway that decreases the level of oxalate in a sample. In one embodiment, the modified bacterium can use oxalate as a sole source of carbon and energy.

The modified bacterium of the disclosure can degrade oxalate, and thus can be used, for example, in carrying out the methods disclosed herein. The inventors described methods for identifying specific mutations that render a bacterium capable of degrading oxalate. These mutations span a number of genes, and the disclosure makes possible identification of other mutations that render a bacterium capable of degrading oxalate, where the starting bacterium was not capable of degrading oxalate or not capable of using oxalate as a sole source of carbon and energy. The examples describe such mutations in the context of E. coli, but those of skill in the art will understand that many bacteria have homologues or orthologues of the proteins disclosed herein with a high percent identity to the corresponding E. coli protein.

In one embodiment, the one or more mutations comprise mutations in one or more genes selected from the group consisting of eutE, eutN, adhE, icd, sthA, pka, aceK, nadR, iclR, rpoB, mg, tosA, and rpoS, all based on naming convention for E. coli; or homologues or orthologues thereof. The full E. coli naming convention for the proteins encoded by these genes is provided in Table 1.

TABLE 1

eutE: Acetaldehyde dehydrogenase
eutN: Bacterial microcompartment vertex protein
adhE: Aldehyde-alcohol dehydrogenase
icd: Isocitrate dehydrogenase
sthA: Soluble pyridine nucleotide transhydrogenase
aceK: Isocitrate dehydrogenase kinase/phosphatase
nadR: Trifunctional NAD biosynthesis/regulator protein
iclR: Transcriptional repressor IclR
rpoB: DNA-directed RNA polymerase subunit beta
rng: Ribonuclease G
rpoS: RNA polymerase sigma factor
pka: protein lysine acetyltransferase
tosA: type 1 secretion A

As shown in the examples that follow, the inventors have identified mutations in the proteins encoded by each of these genes in modified bacterium capable of degrading oxalate. In one embodiment, the one or more mutations comprise mutations in one or more genes selected from the group consisting of eutN, adhE, sthA, and nadR. The inventors have identified mutations in the proteins encoded by each of these genes that by themselves render the modified bacterium expressing the mutated proteins capable of degrading oxalate.

In a further embodiment, the one or more mutations comprise mutations in one or more genes selected from the group consisting of eutN, adhE, and eutE. The inventors have identified mutations in the proteins encoded by each of these genes to be most prevalent in modified bacterium capable of degrading oxalate.

In another embodiment, the one or more mutations comprise mutations in one or more of the following combination of genes:
(a) sthA and adhE;
(b) nadR and eutE;
(c) adhE and icd;
(d) adhE and rpoS;
(e) adhE and rng;
(f) adhE and tosA;
(g) eutN and eutE; and/or
(h) eutN and iclR.

The inventors have identified mutations in the pair of proteins encoded by each of these gene combinations as rendering the modified bacterium capable of degrading oxalate.

In another embodiment, the one or more mutations comprise mutations in one or more of the following combination of genes:
(a) nadR, eutE, and eutN;
(b) adhE, aceK, and pka; and/or
(c) eutN, eutE, and rpoB.

The inventors have identified mutations in the set of proteins encoded by each of these gene combinations as rendering the modified bacterium capable of degrading oxalate.

In one embodiment, one or more mutations comprise one or more mutations in or near an alcohol dehydrogenase domain-encoding portion of the adhE gene, or at position 77. In E. coli, residues 541-840 constitute the alcohol dehydrogenase domain of the AdhE protein, highlighted below in SEQ ID NO:1.

| (SEQ ID NO: 1) | | |
|---|---|---|
| 1 | MAVTNVAELNALVERVKKAQREYASFTQEQVDKIFRAAALAAADARIPLAKMAVAESGMG | 61 |
| 60 | IVEDKVIKNHFASEYIYNAYKDEKTCGVLSEDDTFGTITIAEPIGIICGIVPTTNPTSTA | 120 |
| 121 | IFKSLISLKTRNAIIFSPHPRAKDATNKAADIVLQAAIAAGAPKDLIGWIDQPSVELSNA | 180 |
| 181 | LMHHPDINLILATGGPGMVKAAYSSGKPAIGVGAGNTPVVIDETADIKRAVASVLMSKTF | 240 |
| 241 | DNGVICASEQSVVVVDSVYDAVRERFATHGGYLLQGKELKAVQDVILKNGALNAAIVGQP | 300 |
| 301 | AYKIAELAGFSVPENTKILIGEVTVVDESEPFAHEKLSPTLAMYRAKDFEDAVEKAEKLV | 360 |
| 361 | AMGGIGHTSCLYTDQDNQPARVSYFGQKMKTARILINTPASQGGIGDLYNFKLAPSLTLG | 420 |
| 421 | CGSWGGNSISENVGPKHLINKKTVAKRAENMLWHKLPKSIYFRRGSLPIALDEVITDGHK | 480 |
| 481 | RALIVTDRFLFNNGYADQITSVLKAAGVETEVFFEVEADPTLSIVRKGAELANSFKPDVI | 540 |
| 541 | IALGGGSPMDAAKIMWVMYEHPETHFEELALRFMDIRKRIYKFPKMGVKAKMIAVTTTSG | 600 |
| 601 | TGSEVTPFAVVTDDATGQKYPLADYALTPDMAIVDANLVMDMPKSLCAFGGLDAVTHAME | 660 |
| 661 | AYVSVLASEFSDGQALQALKLLKEYLPASYHEGSKNPVARERVHSAATIAGIAFANAFLG | 720 |
| 721 | VCHSMAHKLGSQFHIPHGLANALLICNVIRYNANDNPTKQTAFSQYDRPQARRRYAEIAD | 780 |
| 781 | HLGLSAPGDRTAAKIEKLLAWLETLKAELGIPKSIREAGVQEADFLANVDKLSEDAFDDQ | 840 |
| 841 | CTGANPRYPLISELKQILLDTYYGRDYVEGETAAKKEAAPAKAEKKAKKSA* | 892 |

As discussed in the examples, observed mutations in AdhE, a cytosolic bifunctional ethanol and acetaldehyde dehydrogenase, cluster around the active site cleft. While not being bound by a specific mechanism of action, these mutations might also increase accessibility to non-native substrates for promiscuous reactions, such as affinity or activity toward oxalate or oxalyl-CoA. Position 77 is at the interface of AdhE's aldehyde-dehydrogenase (AldDH) domain and the ADH domain's alcohol-binding cleft. In one embodiment, the one or more mutations in or near the alcohol dehydrogenase domain-encoding portion of the adhE gene result in the adhE gene encoding an AdhE protein comprising mutations at one or more of residues 77, 546, 581, 600, and 837, numbering based on the E. coli AdhE protein sequence of SEQ ID NO:1. In another embodiment, the one or more mutations in the alcohol dehydrogenase domain-encoding portion of the adhE gene result in the adhE gene encoding an AdhE protein comprising 1, 2, 3, 4, or all 5 mutations selected from Y77H, E546D, Y581C, G600D, and/or F837L, numbering and residues based on the E. coli AdhE protein sequence of SEQ ID NO:1. In a further embodiment, the one or more mutations in or near the alcohol dehydrogenase domain-encoding portion of the adhE gene result in the adhE gene encoding an AdhE protein comprising 2, 3, 4, 5, or all 6 mutations selected from Y77H, E546D, Y581C, F583L, G600D, and/or F837L, numbering and residues based on the E. coli AdhE protein sequence of SEQ ID NO:1.

In another embodiment, the one or more mutations comprise one or more mutations in a signal sequence-encoding portion of the eutE gene. In E. coli, residues 6-14 constitute the signal sequence of the EutE protein, highlighted in SEQ ID NO:2.

| (SEQ ID NO: 2) | | |
|---|---|---|
| 1 | MNQQDIEQVVKAVLLKMQSSDTPSAAVHEMGVFASLDDAVAAAKVAQQGLKSVAMRQLAI | 60 |
| 61 | AAIREAGEKHARDLAELAVSETGMGRVEDKFAKNVAQARGTPGVECLSPQVLTGDNGLTL | 120 |
| 121 | IENAPWGVVASVTPSTNPAATVINNAISLIAAGNSVIFAPHPAAKKVSQRAITLLNQAIV | 180 |
| 181 | AAGGPENLLVTVANPDIETAQRLFKFPGIGLLVVTGGEAVVEAARKHTNKRLIAAGAGNP | 240 |
| 241 | PVVVDETADLARAAQSIVKGASFDNNIICADEKVLIVVDSVADELMRLMEGQHAVKLTAE | 300 |
| 301 | QAQQLQPVLLKNIDERGKGTVSRDWVGRDAGKIAAAIGLKVPQETRLLFVETTAEHPFAV | 360 |
| 361 | TELMMPVLPVVRVANVADAIALAVKLEGGCHHTAAMHSRNIENMNQMANAIDTSIFVKNG | 420 |
| 421 | PCIAGLGLGGEGWTTMTITTPTGEGVTSARTFVRLRRCVLVDAFRIV* | 468 |

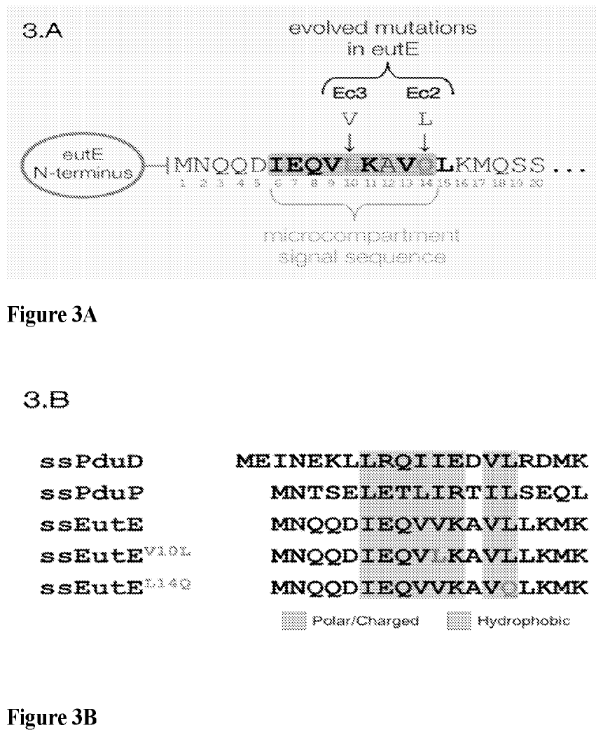

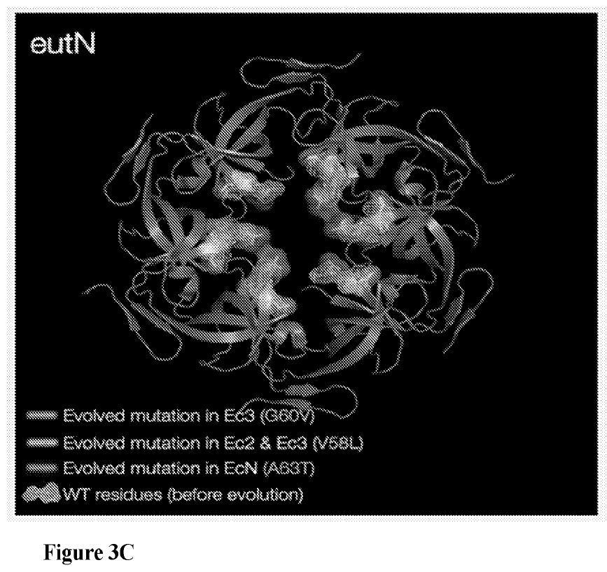

As discussed in the examples, EutE protein is natively localized to the lumen of the ethanolamine-utilization microcompartment (Eut), a bacterial organelle enclosed in a proteinaceous shell that is selectively permeable to the cytosol. The localization of Eut lumen proteins is mediated by N-terminal signal sequences of nine amino acids. While not being bound by any mechanism of action, mutations in the signal sequence may abrogate packaging of EutE into the Eut microcompartment, consistent with the observed putative promiscuous activity of EutE toward cytosolic oxalate or oxalyl-CoA (FIG. 3A+B).

In one embodiment, the one or more mutations comprise a deletion of all or a portion of the eutE gene encoding the signal sequence. In another embodiment, the one or more mutations in the signal sequence-encoding portion of the eutE gene result in the eutE gene encoding a EutE protein comprising mutations at one or both of residues 10 and 14, based on residue numbering for E. coli eutE of SEQ ID NO:2. In one such embodiment, the mutations at one or both of residues 10 and 14 comprise one or both of mutations V10L and/or L14Q mutations, numbering and residues based on the E. coli EutE protein sequence of SEQ ID NO:2.

In a further embodiment, the one or more mutations comprise one or more mutations in the eutN gene encoding pore-facing amino acid residues.

In E. coli, residues 54-81 constitute the pore-facing residues of the EutN protein, highlighted in SEQ ID NO:3.

| (SEQ ID NO: 3) | | |
|---|---|---|
| 1 | MKLAVVTGQIVCTVRHHGLAHDKLLMVEMIDPQGNPDGQCAVAIDNIGAG | 50 |
| 51 | TGEWVLLVSGSSARQAHKSETSPVDLCVIGIVDEVVSGGQVIFHK* | 96 |

As discussed in the examples, and without being bound by a specific mechanism of action, permeability of the Eut microcompartment to the cytosol may be determined by the central pores of its shell proteins, which include EutN. Alterations to the permeability of the Eut microcompartment mediated by EutN pore mutations may increase exposure of EutE to cytosolic oxalate or oxalyl-CoA. Alternatively, observed mutations in EutN might compromise shell formation and release EutE into the cytosol.

In one embodiment, the one or more mutations comprise 1, 2, or all 3 mutations in the eutN gene encoding pore-facing amino acid residues 58, 60, and 63, numbering and residues based on the E. coli EutN protein sequence of SEQ ID NO:3. In another embodiment, the one or more mutations result in the eutN gene encoding a EutN protein comprising 1, 2, or all 3 mutations selected from V58L, G60V, and A63T, numbering and residues based on the E. coli EutN protein sequence of SEQ ID NO:3.

In a further embodiment, the one or more mutations comprise one or more mutations in the nadR gene. In one such embodiment, the one or more mutations result in the nadR gene encoding a NadR protein comprising a mutation at residue 403, numbering and residues based on the E. coli NadR protein sequence of SEQ ID NO:4. In another embodiment, the mutation at residue 403 comprises a R403L mutation.

While not being bound by any specific mechanism, the mutated NadR protein may increase repression of genes involved in NAD synthesis, and thus regenerate redox potential.

| NadR protein sequence (E. coli) | | |
|---|---|---|
| (SEQ ID NO: 4) | | |
| 1 | MSSFDYLKTAIKQQGCTLQQVADASGMTKGYLSQLLNAKIKSPSAQKLEALHRFLGLEFP | 60 |
| 61 | RQKKTIGVVFGKFYPLHTGHIYLIQRACSQVDELHIIMGFDDTRDRALFEDSAMSQQPTV | 120 |
| 121 | PDRLRWLLQTFKYQKNIRIHAFNEEGMEPYPHGWDVWSNGIKKEMAEKGIQPDLIYTSEE | 180 |
| 181 | ADAPQYMEHLGIETVLVDPKRTFMSISGAQIRENPFRYWEYIPTEVKPFFVRTVAILGGE | 240 |
| 241 | SSGKSTLVNKLANIFNTTSAWEYGRDYVFSHLGGDEIALQYSDYDKIALGHAQYIDFAVK | 300 |
| 301 | YANKVAFIDTDFVTTQAFCKKYEGREHPFVQALIDEYRFDLVILLENNTPWVADGLRSLG | 360 |
| 361 | SSVDRKEFQNLLVEMLEENNIEFVRVEEEDYDSRFLRCVELVREMMGEQR* | 411 |

In one embodiment, the one or more mutations comprise one or more mutations in the sthA gene. In one such embodiment, the one or more mutations result in the sthA gene encoding a SthA protein comprising a mutation at one or both of residues 150 and 268, numbering and residues based on the E. coli NadR protein sequence of SEQ ID NO:5. In a further embodiment, the mutation at one or both of residues 150 and 268 comprises P150T and/or A268T mutations. In one embodiment, the one or more mutations result in the sthA gene encoding a SthA protein comprising a P150T mutation, numbering and residues based on the E. coli SthA protein sequence of SEQ ID NO:5.

| SthA protein sequence (E. coli) | | |
|---|---|---|
| (SEQ ID NO: 5) | | |
| 1 | MPHSYDYDAIVIGSGPGGEGAAMGLVKQGARVAVIERYQNVGGGCTHWGTIPSKALRHAV | 60 |
| 61 | SRIIEFNQNPLYSDHSRLLRSSFADILNHADNVINQQTRMRQGFYERNHCEILQGNARFV | 120 |
| 121 | DEHTLALDCPDGSVETLTAEKFVIACGSRPYHPTDVDFTHPRIYDSDSILSMHHEPRHVL | 180 |
| 181 | IYGAGVIGCEYASIFRGMDVKVDLINTRDRLLAFLDQEMSDSLSYHFWNSGVVIRHNEEY | 240 |
| 241 | EKIEGCDDGVIMHLKSGKKLKADCLLYANGRTGNTDSLALQNIGLETDSRGQLKVNSMYQ | 300 |
| 301 | TAQPHVYAVGDVIGYPSLASAAYDQGRIAAQALVKGEATAHLIEDIPTGIYTIPEISSVG | 360 |
| 361 | KTEQQLTAMKVPYEVGRAQFKHLARAQIVGMNVGTLKILFHRETKEILGIHCFGERAAEI | 420 |
| 421 | IHIGQAIMEQKGGGNTIEYFVNTTFNYPTMAEAYRVAALNGLNRLF* | 467 |

While not being bound by any specific mechanism, the mutated SthA protein may help regenerate the redox potential of the oxalate utilization pathway.

In various further embodiments, the modified bacterium comprises one or more mutations that result in genes encoding one or more sets of mutated proteins selected from the group consisting of the following, each of which is exemplified in the examples, numbering and residues based on the corresponding E. coli amino acid sequences disclosed herein.
(a) SthA P150T;
(b) AdhE F583L and Icd D158G;
(c) AdhE F583L and SthA A268T;
(d) AdhE F583L, AceK R565C, and Pka G596W;
(e) NadR R403L;
(f) EutE L14Q, EutN V58L, and NadR R403L:
(g) EutE L14Q and EutN V58L;
(h) EutE L14Q and NadR R403L;
(i) AdhE Y581C;
(j) AdhE Y581C and AdhE G546D;
(k) EutN G60V;
(l) EutN V58L, EutN G60V, EutE V10L;
(m) EutN G60V and a knockout or truncation mutation of IclR, including but not limited to Q137*, wherein the asterisk represents a truncation at position 137;
(n) EutN G60V, EutE V10L, and RpoB A532E;
(o) AdhE Y77H and RpoS G66V;
(p) AdhE Y77H;
(q) EutN A63 T;
(r) AdhE G600D and Rng R394P;
(s) AdhE Y77H and TosA Y919C; and
(t) AdhE F837L, AdhE Y77H, and TosA Y919C.

In another embodiment, the modified bacterium comprises a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 1, and including 1, 2, 3, 4, or all 5 residues selected from 77H, 546D, 581C, 600D, and/or F837L.

In a further embodiment, the modified bacterium comprises a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 1, and including 2, 3, 4, 5, or all 6 residues selected from 77H, 6546D, 581C, F583L, 600D, and/or F837L.

In one embodiment, the modified bacterium comprises a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 2, and including one both residues selected from 10L and 14Q.

In another embodiment, the modified bacterium comprises a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 3, and including 1, 2, or all 3 residues selected from 58L, 60V, and 63T.

In a further embodiment, the modified bacterium comprises a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 4, and including residue 403L.

In another embodiment, the modified bacterium comprises a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 5 and including residue 150T and/or A268T.

As disclosed in the examples that follow, mutations in other genes and their encoded proteins were identified in the modified bacteria. Thus, in another embodiment, the modified bacterium may comprise a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of one or more of the following:
(a) SEQ ID NO: 6 and including residue D158G (IcD);
(b) SEQ ID NO:7 and including residue R565C (AceK);
(c) SEQ ID NO:8 truncated at residue Q137* (IclR), wherein the asterisk represents a truncation of the protein;
(d) SEQ ID NO:9 and including residue A532E (RpoB);
(e) SEQ ID NO:10 and including residue G66V (RpoS);
(f) SEQ ID NO:11 and including residue R394P (Rng);
(g) SEQ ID NO:12 and including residue tosA Y919C (TosA); and/or
(h) SEQ ID NO:13 and including residue pka G596W (Pka).

UniProtKB-P08200 (IDH_ECOLI) Isocitrate dehydrogenase icd [NADP]
>sp|P08200|IDH_ECOLI Isocitrate dehydrogenase [NADP] OS = Escherichia coli
(strain K12) OX = 83333 GN = icd PE = 1 SV = 1
(SEQ ID NO: 6)
MESKVVVPAQGKKITLQNGKLNVPENPIIPYIEGDGIGVDVTPAMLKVVDAAVEKAYKGE
RKISWMEIYTGEKSTQVYGQDVWLPAETLDLIREYRVAIKGPLTTPVGGGIRSLNVALRQ
ELDLYICLRPVRYYQGTPSPVKHPELTDMVIFRENSEDIYAGIEWKADSADAEKVIKFLR
EEMGVKKIRFPEHCGIGIKPCSEEGTKRLVRAAIEYAIANDRDSVTLVHKGNIMKFTEGA
FKDWGYQLAREEFGGELIDGGPWLKVKNPNTGKEIVIKDVIADAFLQQILLRPAEYDVIA
CMNLNGDYISDALAAQVGGIGIAPGANIGDECALFEATHGTAPKYAGQDKVNPGSIILSA
EMMLRHMGWTEAADLIVKGMEGAINAKTVTYDFERLMDGAKLLKCSEFGDAIIENM
UniProtKB-P11071 (ACEK_ECOLI) Isocitrate dehydrogenase kinase/phosphatase
>sp|P11071|ACEK_ECOLI Isocitrate dehydrogenase kinase/phosphatase
OS = Escherichia coli (strain K12) OX = 83333 GN = aceK PE = 1 SV = 2
(SEQ ID NO: 7)
MPRGLELLIAQTILQGFDAQYGRFLEVTSGAQQRFEQADWHAVQQAMKNRIHLYDHHVGL
VVEQLRCITNGQSTDAAFLLRVKEHYTRLLPDYPRFEIAESFFNSVYCRLFDHRSLTPER
LFIFSSQPERRFRTIPRPLAKDFHPDHGWESLLMRVISDLPLRLRWQNKSRDIHYIIRHL
TETLGTDNLAESHLQVANELFYRNKAAWLVGKLITPSGTLPFLLPIHQTDDGELFIDTCL
TTTAEASIVFGFARSYFMVYAPLPAALVEWLREILPGKTTAELYMAIGCQKHAKTESYRE
YLVYLQGCNEQFIEAPGIRGMVMLVFTLPGFDRVFKVIKDRFAPQKEMSAAHVRACYQLV
KEHDRVGRMADTQEFENFVLEKRHISPALMELLLQEAAEKITDLGEQIVIRHLYIERRMV
PLNIWLEQVEGQQLRDAIEEYGNAIRQLAAANIFPGDMLFKNFGVTRHGRVVFYDYDEIC
YMTEVNFRDIPPPRYPEDELASEPWYSVSPGDVFPEEFRHWLCADPRIGPLFEEMHADLF
RADYWRALQNRIREGHVEDVYAYRRRQRFSVRYGEMLF
UniProtKB-P16528 (ICLR_ECOLI) Transcriptional repressor IclR
>sp|P16528|ICLR_ECOLI Transcriptional repressor IclR OS = Escherichia coli
(strain K12) OX = 83333 GN = iclR PE = 1 SV = 1
(SEQ ID NO: 8)
MVAPIPAKRGRKPAVATAPATGQVQSLTRGLKLLEWIAESNGSVALTELAQQAGLPNSTT
HRLLTTMQQQGFVRQVGELGHWAIGAHAFMVGSSFLQSRNLLAIVHPILRNLMEESGETV
NMAVLDQSDHEAIIIDQVQCTHLMRMSAPIGGKLPMHASGAGKAFLAQLSEEQVTKLLHR
KGLHAYTHATLVSPVHLKEDLAQTRKRGYSFDDEEHALGLRCLAACIFDEHREPFAAISI
SGPISRITDDRVTEFGAMVIKAAKEVTLAYGGMR
UniProtKB-P0A8V2 (RPOB_ECOLI) DNA-directed RNA polymerase subunit beta
>sp|POA8V2|RPOB_ECOLI DNA-directed RNA polymerase subunit beta
OS = Escherichia coli (strain K12) OX = 83333 GN = rpoB PE = 1 SV = 1
(SEQ ID NO: 9)
MVYSYTEKKRIRKDFGKRPQVLDVPYLLSIQLDSFQKFIEQDPEGQYGLEAAFRSVFPIQ
SYSGNSELQYVSYRLGEPVFDVQECQIRGVTYSAPLRVKLRLVIYEREAPEGTVKDIKEQ
EVYMGEIPLMTDNGTFVINGTERVIVSQLHRSPGVFFDSDKGKTHSSGKVLYNARIIPYR
GSWLDFEFDPKDNLFVRIDRRRKLPATIILRALNYTTEQILDLFFEKVIFEIRDNKLQME
LVPERLRGETASFDIEANGKVYVEKGRRITARHIRQLEKDDVKLIEVPVEYIAGKVVAKD
YIDESTGELICAANMELSLDLLAKLSQSGHKRIETLFTNDLDHGPYISETLRVDPTNDRL
SALVEIYRMMRPGEPPTREAAESLFENLFFSEDRYDLSAVGRMKENRSLLREEIEGSGIL
SKDDIIDVMKKLIDIRNGKGEVDDIDHLGNRRIRSVGEMAENQFRVGLVRVERAVKERLS
LGDLDTLMPQDMINAKPISAAVKEFFGSSQLSQFMDQNNPLSEITHKRRISALGPGGLTR
ERAGFEVRDVHPTHYGRVCPIETPEGPNIGLINSLSVYAQTNEYGFLETPYRKVTDGVVT
DEIHYLSAIEEGNYVIAQANSNLDEEGHFVEDLVTCRSKGESSLESRDQVDYMDVSTQQV
VSVGASLIPFLEHDDANRALMGANMQRQAVPTLRADKPLVGTGMERAVAVDSGVTAVAKR
GGVVQYVDASRIVIKVNEDEMYPGEAGIDIYNLTKYTRSNQNTCINQMPCVSLGEPVERG
DVLADGPSTDLGELALGQNMRVAFMPWNGYNFEDSILVSERVVQEDRFTTIHIQELACVS
RDTKLGPEEITADIPNVGEAALSKLDESGIVYIGAEVTGGDILVGKVTPKGETQLTPEEK
LLRAIFGEKASDVKDSSLRVPNGVSGTVIDVQVFTRDGVEKDKRALEIEEMQLKQAKKDL
SEELQILEAGLFSRIRAVLVAGGVEAEKLDKLPRDRWLELGLTDEEKQNQLEQLAEQYDE
LKHEFEKKLEAKRRKITQGDDLAPGVLKIVKVYLAVKRRIQPGDKMAGRHGNKGVISKIN
PIEDMPYDENGTPVDIVLNPLGVPSRMNIGQILETHLGMAAKGIGDKINAMLKQQQEVAK
LREFIQRAYDLGADVRQKVDLSTFSDEEVMRLAENLRKGMPIATPVFDGAKEAEIKELLK
LGDLPTSGQIRLYDGRTGEQFERPVTVGYMYMLKLNHLVDDKMHARSTGSYSLVTQQPLG
GKAQFGGQRFGEMEVWALEAYGAAYTLQEMLTVKSDDVNGRTKMYKNIVDGNHQMEPGMP
ESFNVLLKEIRSLGINIELEDE
UniProtKB-P13445 (RPOS_ECOLI) RNA polymerase sigma factor RpoS
>sp|P13445|RPOS_ECOLI RNA polymerase sigma factor RpoS OS = Escherichia coli
(strain K12) OX = 83333 GN = rpoS PE = 1 SV = 3
(SEQ ID NO: 10)
MSQNTLKVHDLNEDAEFDENGVEVEDEKALVEQEPSDNDLAEEELLSQGATQRVLDATQL
YLGEIGYSPLLTAEEEVYFARRALRGDVASRRRMIESNLRLVVKIARRYGNRGLALLDLI
EEGNLGLIRAVEKFDPERGFRFSTYATWWIRQTIERAIMNQTRTIRLPIHIVKELNVYLR
TARELSHKLDHEPSAEEIAEQLDKPVDDVSRMLRLNERITSVDTPLGGDSEKALLDILAD
EKENGPEDTTQDDDMKQSIVKWLFELNAKQREVLARRFGLLGYEAATLEDVGREIGLTRE
RVRQIQVEGLRRLREILQTQGLNIEALFRE
UniProtKB-P0A9J0 (RNG_ECOLI) Ribonuclease G
>sp|P0A9J0|RNG_ECOLI Ribonuclease G OS = Escherichia coli (strain K12)
OX = 83333 GN = rng PE = 1 SV = 2
(SEQ ID NO: 11)
MTAELLVNVTPSETRVAYIDGGILQEIHIEREARRGIVGNIYKGRVSRVLPGMQAAFVDI
GLDKAAFLHASDIMPHTECVAGEEQKQFTVRDISELVRQGQDLMVQVVKDPLGTKGARLT
TDITLPSRYLVFMPGASHVGVSQRIESESERERLKKVVAEYCDEQGGFIIRTAAEGVGEA
ELASDAAYLKRVWTKVMERKKRPQTRYQLYGELALAQRVLRDFADAELDRIRVDSRLTYE
ALLEFTSEYIPEMTSKLEHYTGRQPIFDLEDVENEIQRALERKVELKSGGYLIIDQTEAM
TTVDINTGAFVGHRNLDDTIENTNIEATQAIARQLRLRNLGGIIIIDFIDMNNEDHRRRV
LHSLEQALSKDRVKTSVNGFSALGLVEMTRKRTRESIEHVLCNECPTCHGRGTVKTVETV
CYEIMREIVRVHHAYDSDRFLVYASPAVAEALKGEESHSLAEVEIFVGKQVKVQIEPLYN
QEQFDVVMM
TosA
(SEQ ID NO: 12)
MKMIFTGKVSGEKTVLTVGGRHTVKAQPGEQYGLIDEVTGLVPDGVEADRSGDDLILRKKEEDTEVRIEGFWEE
CQPGETQCTAVFNIVGEDGQVTEAVLTQDGPVLDDITAGQSGTLSDDDRGGFIWLGGLAFGGGLAAMALAAGGG
GSKHRHENDDSDTTAPSSPALKAEDDGSVSVELPGDANKGDTVDVTFEDEKGGKHTVTLEKGDNGWTSSDPTLI
PDSTGDKATIPADNVKDNSEVTGVAKDPSGNESDPSTVTSKTDVLPTVSISVETTSTDVNGDGFTGIASVNGTV
MDVPATIEDKDDSTGLVYTVSLNHVTTTDVTVTVTLGSGAGHSDAADYSDIGGAQHNGKIGLHGDTGKVTYDGA
TTVTIVIPAGSKSVSFIVDPTLEANQDAFNAEGMEKVVATITGTSDNVTAATDIVDNAGASATGVIYDGNAISL
RNLDGDFTLKYSLSSSIAEKGDFGYTIGANSGENDPMVTTDYNDTVYVGYYQSGKETTSYSNVANSQDNGPDGT
KTDGNQSITTVDLGAGDDLMVIRGNMLANTRVYTGEGNDTFTMDGMNTALRVMYAGSYIFTESGDDIVTIKRTG
VTNAGQIYLGSGSDTFIQGDATDNNDTTLSGLLDLGSGTKDISNMPKEYLSVYQDGSNLSLGNDNNIDTATDVN
TVTIYGSVSGEILGGYGSDNITVTKNLTGNISVGDNADTLTAGWIYGGSTVSMGDGNDTVTITDGAYNTTISLG
AGDDVFDATSGVMGDSAYATVVNGEDGNDTFKLGTIAKNLTIDAGAGDDIVVLTKDYDSTSSGNQGYINGGEGS
DTLVLTGTISVNLAAGKNEGIAGFEKVDMTVGSDLKADNAAQLVKLTASDVLGINDNSTIYISGDANDKVDLGA
DGAGSLGTFTATATTVKATALDGIEHTYTLYSSVSGANVYIDNNIIDANGVI
Pka
(SEQ ID NO: 13)
MSQRGLEALLRPKSIAVIGASMKPNRAGYLMMRNLLAGGFNGPVLPVTPAWKAVLGVLAWPDIASLPFTPDLAV
LCTNASRNLALLEELGEKGCKTCIILSAPASQHEDLRACALRHNMRLLGPNSLGLLAPWQGLNASFSPVPIKRG
KLAFISQSAAVSNTILDWAQQRKMGFSYFIALGDSLDIDVDELLDYLARDSKTSAILLYLEQLSDARRFVSAAR
SASRNKPILVIKSGRSPAAQRLLNTTAGMDPAWDAAIQRAGLLRVQDTHELFSAVETLSHMRPLRGDRLMIISN
GAAPAALALDALWSRNGKLATLSEETCQKLRDALPEHVAISNPLDLRDDASSEHYIKTLDILLHSQDEDALMVI
HSPSAAAPATESAQVLIEAVKHHPRSKYVSLLTNWCGEHSSQEARRLFSEAGLPTYRTPEGTITAFMHMVEYRR
NQKQLRETPALPSNLTSNTAEAHLLLQQAIAEGATSLDTHEVQPILQAYGMNTLPTWIASDSTEAVHIAEQIGY
PVALKLRSPDIPHKSEVQGVMLYLRTANEVQQAANAIFDRVKMAWPQARVHGLLVQSMANRAGAQELRVVVEHD
PVFGPLIMLGEGGVEWRPEDQAVVALPPLNMNLARYLVIQGIKSKKIRARSALRPLDVAGLSQLLVQVSNLIVD
CPEIQRLDIHPLLASGSEFTALDVTLDISPFEGDNESRLAVRPYPHQLEEWVELKNGERCLFRPILPEDEPQLQ
QFISRVTKEDLYYRYFSEINEFTHEDLANMTQIDYDREMAFVAVRRIDQTEEILGVTRAISDPDNIDAEFAVLV
RSDLKGLGLGRRLMEKLITYTRDHGLQRLNGITMPNNRGMVALARKLGENVDIQLEEGIVGLTLNLAQREES

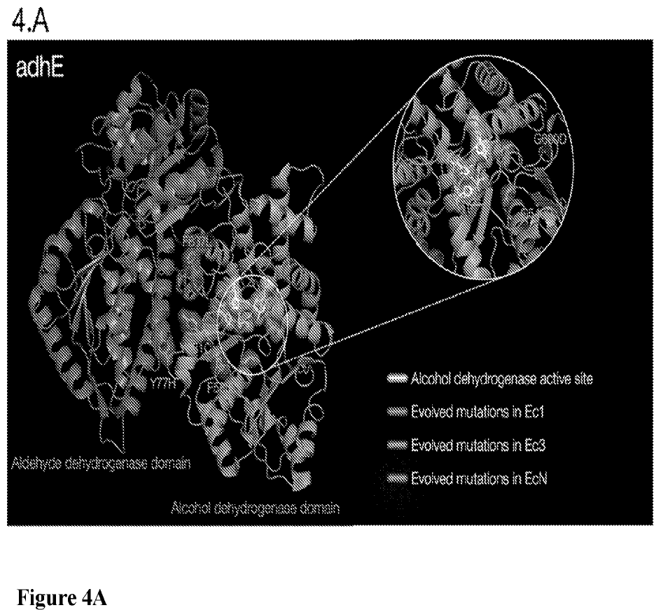

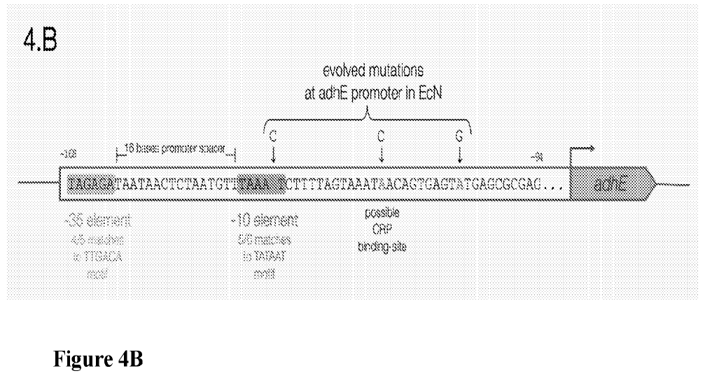

As also described in the examples that follow, mutations in the adhE gene promoter sequence were also identified that increase expression of the adhE gene product. Thus, in another embodiment, the modified bacterium further comprise one or more mutations in the adhE gene promoter sequence. In one embodiment, the one or more mutations in the adhE gene promoter sequence comprise one or more A to C mutations in the region 95 to 150 nucleotides upstream of the adhE coding sequence start codon (based on the E. coli promoter sequence), for example, at nucleotides 104, 114, and/or 128 residues upstream of the start codon. See FIG. 4B.

In one embodiment, the one or more mutations may be generated by subjecting bacteria to artificial conditions that force growth on oxalate, as discussed in the examples. In another embodiment, the bacterium is genetically engineered to express the one or more mutant proteins. In this embodiment, the one or more mutated gene may be operatively linked to a recombinant regulatory sequence. In one embodiment, the recombinant regulatory sequence comprises a heterologous promoter. As used herein, a heterologous promote is a promoter that is not the naturally occurring promoter for the gene in question. Any suitable heterologous promoter may be used as deemed appropriate for an intended use. In one embodiment, the promoter is an inducible promoter. Any suitable inducible promoter may be used as appropriate for an intended purpose. In one embodiment, the inducible promoter is inducible by exogenous environmental conditions found in the mammalian gut, making it particularly useful for mammalian administration. In another embodiment, the inducible promoter is inducible by low-oxygen or anaerobic conditions. In various non-limiting embodiments, wherein the inducible promoter may be selected from the group consisting of an FNR-responsive promoter, an ANR-responsive promoter, and a DNR-responsive promoter (see US20180325963, incorporated by reference herein).

In all these embodiments, the one or more mutated genes may be located on a chromosome in the bacterium, may be located on a plasmid in the bacterium, or combinations thereof.

The modified bacterium may be any bacterium that in its wild-type state does not degrade oxalate, or may be any bacterium that in its wild-type state is not capable of utilizing oxalate as a sole source of carbon and energy. While the examples disclosed herein are based on mutations in E. coli, such as strain EcN (E. coli Nissle 1917), those of skill in the art will understand that the mutated proteins disclosed herein have closely related homologues and orthologues in a wide variety of other bacteria that do not degrade oxalate, and thus the results disclosed can be readily extrapolated to other bacteria Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the modified bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, and Lactococcus lactis.

In one embodiment, the bacterium is a probiotic bacterial cell and non-pathogenic to mammals, such as humans. In other embodiments, the bacterium is a member of a genus selected from the group consisting of Bacteroides, Bifidobacterium, Clostridium, Escherichia, Streptococcus, Lactobacillus and Lactococcus. In various further embodiments, the bacterium is of the genus Escherichia, including but not limited to Escherichia coli, including but not limited to E. coli Nissle 1917.

In various further embodiments, the modified bacterium is capable of:
(a) using oxalate as a sole source of carbon and energy;
(b) degrading oxalate in the absence of any other energy source; and/or
(c) generating ATP by its degrading of oxalate.

In another embodiment, the disclosure provides a bacterial population, comprising a plurality of the modified bacterium of any embodiment or combination of embodiments disclosed herein. The population may comprise a single genus or species of bacteria, or may comprise combinations of modified bacterium. The plurality of modified bacterium may be present in any amount suitable for an intended use. In one non-limiting embodiment, the plurality of modified bacterium are present in the population at between about 10⁴ and 10¹² colony forming units (cfu). The population may be provided in any form suitable for an intended purpose. In various embodiments, the population may be present in solution, plated on media, frozen, or dehydrated, such as a lyophilized population. The population may comprise other non-modified (as “modified” is used herein) bacteria, such as non-modified probiotic bacteria, as appropriate for an intended use.

In another aspect, the disclosure provides isolated proteins, comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence selected from the following:
(a) SEQ ID NO: 1, and including 1, 2, 3, 4, or all 5 residues selected from 77H, 546D, 581C, 600D, and/or F837L;
(b) SEQ ID NO: 1, and including 2, 3, 4, 5, or all 6 residues selected from 77H, 546D, 581C, F583L, 600D, and/or F837L;
(c) SEQ ID NO: 2, and including one or both residues selected from 10L and 14Q;
(d) SEQ ID NO: 3, and including 1, 2, or all 3 residues selected from 58L, 60V, and 63T;
(e) SEQ ID NO: 4, and including residue 403L;
(f) SEQ ID NO: 5 and including residue 150T and/or A268T;
(g) SEQ ID NO: 6 and including residue D158G;
(h) SEQ ID NO:7 and including residue R565C;
(i) SEQ ID NO:8 and including residue Q137*, wherein the asterisk represents a truncation of the protein;
(j) SEQ ID NO:9 and including residue A532E;
(k) SEQ ID NO:10 and including residue G66V;
(l) SEQ ID NO:11 and including residue R394P;
(m) SEQ ID NO:12 and including residue Y919C; and
(n) SEQ ID NO:13 and including residue G596W.

In one embodiment the protein comprises acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence selected from the following:
(a) SEQ ID NO: 1, and including 1, 2, 3, 4, or all 5 residues selected from 77H, 546D, 581C, 600D, and/or F837L;
(b) SEQ ID NO: 1, and including 2, 3, 4, 5, or all 6 residues selected from 77H, 546D, 581C, F583L, 600D, and/or F837L;
(c) SEQ ID NO: 2, and including one or both residues selected from 10L and 14Q;
(d) SEQ ID NO: 3, and including 1, 2, or all 3 residues selected from 58L, 60V, and 63T;
(e) SEQ ID NO: 4, and including residue 403L;
(f) SEQ ID NO: 5 and including residue 150T and/or A268T; or including residue 150T.

In another aspect, the disclosure provides nucleic acid encoding the polypeptide of any embodiment disclosed herein. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, termination signals, and ribosome binding sites. Such expression vectors can be of any type, including but not limited plasmid and viral-based expression vectors. The control sequence used to drive expression of the disclosed nucleic acid sequences may be constitutive or inducible (driven by any of a number of inducible promoters including, but not limited to, those disclosed above; antibiotic inducible promoters, etc.) The expression vector must be replicable in the host organism either as an episome or by integration into host chromosomal DNA. In various embodiments, the expression vector may comprise a plasmid.

In another aspect, the disclosure provides host cells that comprise the proteins, nucleic acids, and/or expression vectors (i.e.: episomal or chromosomally integrated), disclosed herein. The host cells may be prokaryotic cells; in one embodiment the host cells are the modified bacteria of the disclosure.

In one embodiment, the disclosure provides composition comprising the modified bacterium, bacterial population, polypeptide, nucleic acid, expression vector, and/or host cell of any embodiment or combination of embodiments disclosed herein. In some embodiments, the compositions may be formulated as pharmaceutical compositions. In other embodiments, the compositions may be comprised in food or beverages.

As used herein a “pharmaceutical composition” comprises a composition of the disclosure, with other components such as a physiologically suitable carrier and/or excipient. The pharmaceutically acceptable carrier is any carrier or a diluent that does not cause significant irritation to an organism and does not interfere with the activity of the composition.

Pharmaceutical compositions of the disclosure may be formulated using one or more physiologically acceptable carriers comprising excipients and auxiliaries, which facilitate processing of the active ingredients into compositions for pharmaceutical use. In some embodiments, the pharmaceutical compositions are subjected to standard processing techniques to form tablets, granulates, nanoparticles, nanocapsules, microcapsules, microtablets, pellets, or powders, which may be enterically coated or uncoated.

The compositions may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the modified bacteria may range from about 10⁴ to 10¹² colony bacteria or colony forming units. The composition may be administered once or more daily, weekly, or monthly. The composition may be administered before, during, or following a meal.

The compositions may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the compositions may be formulated in a solution of sodium bicarbonate, e.g., 1 molar solution of sodium bicarbonate (to buffer an acidic cellular environment, such as the stomach, for example). The compositions may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The compositions may formulated for oral use as tablets, pills, capsules, liquids, gels, syrups, slurries, or suspensions. Pharmacological compositions for oral use can be made using a solid excipient. Suitable excipients may include, fillers such as sugars (lactose, sucrose, mannitol, sorbitol, etc.); cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

In some embodiments, the compositions are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels. Materials used for enteric coatings include Cellulose acetate phthalate (CAP), Poly(methacrylic acid-co-methyl methacrylate), Cellulose acetate trimellitate (CAT), Poly(vinyl acetate phthalate) (PVAP) and Hydroxypropyl methylcellulose phthalate (HPMCP), fatty acids, waxes, Shellac (esters of aleurtic acid), plastics and plant fibers. Additionally, Zein, Aqua-Zein (an aqueous zein formulation containing no alcohol), amylose starch and starch derivatives, and dextrins (e.g., maltodextrin) are also used. Other known enteric coatings include ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate.

In one embodiment, the composition may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In another embodiment, the compositions may be a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods, nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the compositions are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. In yet another embodiment, the composition is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents. In another aspect, the disclosure provides methods for reducing oxalate, comprising administering to a subject in need thereof an amount effective to reduce oxalate of the modified bacterium, bacterial population, polypeptide, nucleic acid, expression vector, host cell, and/or composition of any embodiment or combination of embodiments of the disclosure. In another aspect, the disclosure provides methods for treating or limiting development of a disorder in which oxalate is detrimental in a subject, comprising administering to a subject in need thereof an amount effective to treat or limit development of the disorder of the modified bacterium, bacterial population, polypeptide, nucleic acid, expression vector, host cell, and/or composition of any embodiment or combination of embodiments of the disclosure. In a further embodiment, the disclosure provides methods for treating or limiting development of hyperoxaluria, comprising administering to a subject having hyperoxaluria or at risk of developing hyperoxaluria an amount effective to treat or limit development of hyperoxaluria of the modified bacterium, bacterial population, polypeptide, nucleic acid, expression vector, host cell, and/or composition of any embodiment or combination of embodiments of the disclosure.

Administrative routes are discussed above. In one embodiment, the subject has hyperoxaluria or is at risk of developing hyperoxaluria.

Hyperoxaluria is characterized by urinary oxalate excretion exceeding 40-45 mg per 24 hours. Untreated, hyperoxaluria can cause significant morbidity and mortality, including the development of renal stones (kidney stones), nephrocalcinosis (increased calcium in the kidney) and most significantly, End Stage Renal Disease. Primary hyperoxalurias are autosomal-recessive inherited diseases resulting from mutations in one of several genes involved in oxalate metabolism (Hoppe et al., Nephr. Dial. Transplant. 26: 3609-15 (2011)). Primary hyperoxalurias are characterized by elevated urinary oxalate excretion that ultimately may result in recurrent urolithiasis, progressive nephrocalcinosis and early end-stage renal disease. In addition, when chronic renal insufficiency occurs in patients with primary hyperoxalurias, systemic deposition of calcium oxalate (also known as oxalosis) may occur in various organ systems which can lead to bone disease, erythropoietin refractory anemia, skin ulcers, digital gangrene, cardiac arrhythmias, and cardiomyopathy.

Secondary hyperoxaluria typically results from conditions underlying increased absorption of oxalate, including increased dietary intake of oxalate, increased intestinal absorption of oxalate, excessive intake of oxalate precursors, gut microflora imbalances, and genetic variations of intestinal oxalate transporters. Increased oxalate absorption with consequent hyperoxaluria, often referred to as enteric hyperoxaluria, is observed in patients with a variety of intestinal disorders, including the syndrome of bacterial overgrowth, Crohn's disease, inflammatory bowel disease, as well as other malabsorptive states, such as, after jejunoileal bypass for obesity, after gastric ulcer surgery, and chronic mesenteric ischemia. In addition, hyperoxaluria may also occur following renal transplantation. Each of these is thus a risk factor for developing hyperoxaluria. Patients with secondary hyperoxalurias and enteric hyperoxalurias are predisposed to developing calcium oxalate stones, which may lead to significant renal damage and ultimately result in End Stage Renal Disease.

In one embodiment, the hyperoxaluria is selected from primary hyperoxaluria type I, primary hyperoxaluria type II, primary hyperoxaluria type III, enteric hyperoxaluria, dietary hyperoxaluria, idiopathic hyperoxaluria. In another embodiment, the methods comprise methods to treat or limit development of kidney stones.

The disclosure also provides methods of producing bacteria that can degrade oxalate, comprising
(a) culturing a bacterial population in an anaerobic chamber in a medium comprising oxalate supplemented with glycerol;
(b) performing 1:100 dilutions of the population into fresh medium every 48 hours; and
(c) every 10 dilutions, plating samples of the population onto agar comprising oxalate to detect mutants capable of degrading oxalate.

In one embodiment, glycerol is omitted from the medium once samples from that population exhibit colonies on plates containing only oxalate. Other details of the method of this aspect are disclosed in the examples that follow.

### Examples

Controlling the metabolic repertoires of microbes holds immense industrial and medical potential. For example, microbes that can degrade oxalate (C₂O₄²⁻) in the gut are a promising treatment for kidney stone disease, as colonic oxalate is a primary constituent of most kidney stones. Here we show that gut commensals can evolve de novo oxalate degradation during just 400 generations of propagation in oxalate-enriched, nutrient-poor medium. We evolved three E. coli directly isolated from human volunteers and the probiotic E. coli Nissle 1917 under microaerobic conditions that mimic the gut. While no wild E. coli is known to utilize oxalate, our isolates evolved robust growth on oxalate as a sole source of carbon and energy. We performed whole-genome sequencing and identified three genes repeatedly mutated across strains: adhE, eutE, and eutN. AdhE and EutE are paralogous aldehyde dehydrogenases, while EutN is a structural protein enclosing EutE. Mutations in these three genes suggest convergent mechanisms for de novo oxalate catabolism. Overall, our work shows that laboratory evolution can uncover efficient mechanisms for de novo metabolism of target molecules that could not be obtained or predicted otherwise. Moreover, laboratory evolution is an efficient technique for introducing useful pathways in human gut isolates.

Kidney stone disease has so far eluded successful treatment with exogenous functional probiotics. Most kidney stones are composed of calcium oxalate, which accumulates from both endogenous synthesis and the diet. Therefore, patients with calcium oxalate kidney stone disease are often prescribed low-oxalate diets. Here we evolve four genetically diverse human fecal E. coli isolates to grow on oxalate as a sole source of carbon and energy in gut-like microaerobic conditions, demonstrating that laboratory evolution can be harnessed to obtain potentially therapeutic functions in gut microbes.

### Results

E. coli Gut Isolates Evolve De Novo Oxalate Utilization in Microaerobic Conditions.

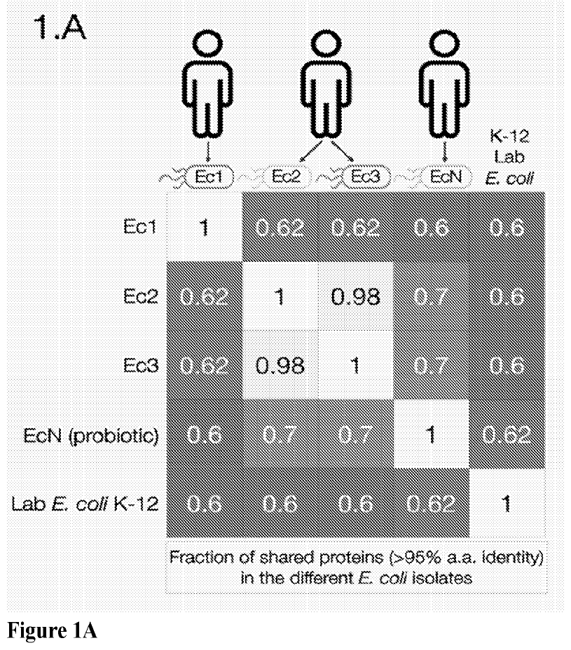

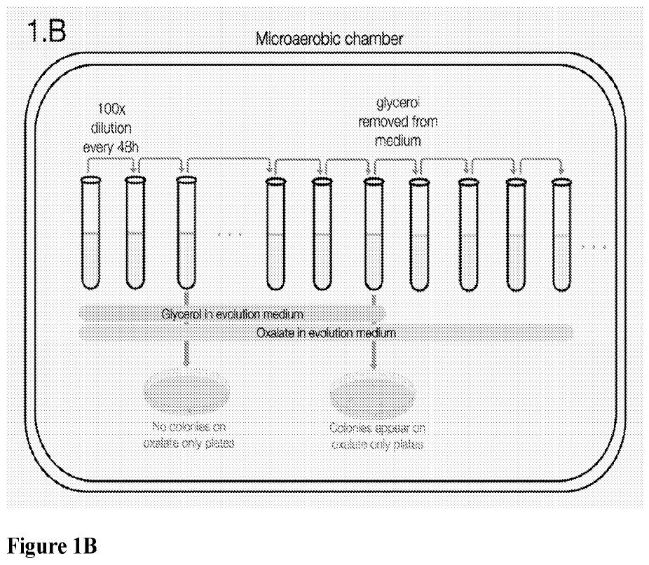

We obtained four E. coli isolates from different backgrounds: strains Ec1, Ec2, and Ec3, isolated from healthy human volunteer stool; and strain EcN (E. coli Nissle 1917), a well-characterized, widely-used probiotic also originally isolated from human stool. Whole genome sequencing of these strains showed high genetic diversity, except between Ec2 and Ec3, which were isolated from the same volunteer and thus expected to show high genetic similarity (FIG. 1A). We focused on E. coli because, to our knowledge, no reports indicate that any wild E. coli strain can utilize oxalate as a sole source of carbon and energy. Indeed, none of our four isolates exhibits growth on oxalate as a sole source of carbon and energy in aerobic, microaerobic, or anaerobic conditions. Furthermore, EcN has been used as a base strain in recent work on recombinant microbiome therapeutics for phenylketonuria and other diseases.

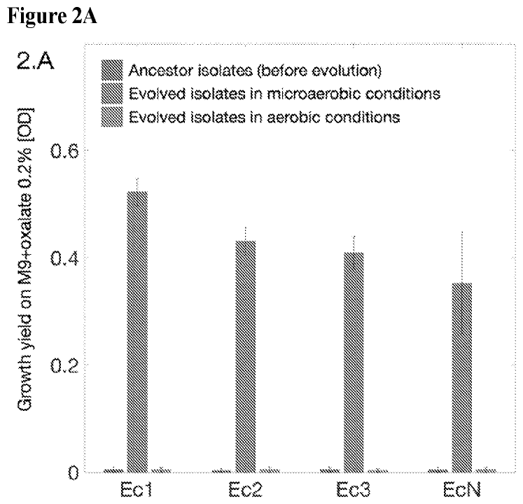

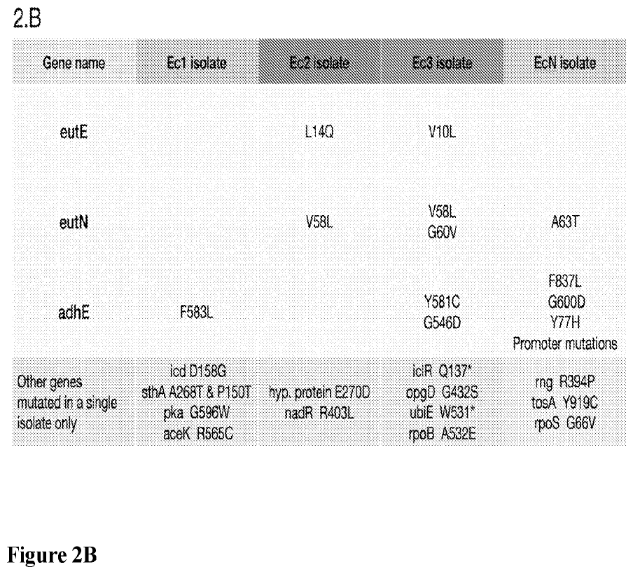

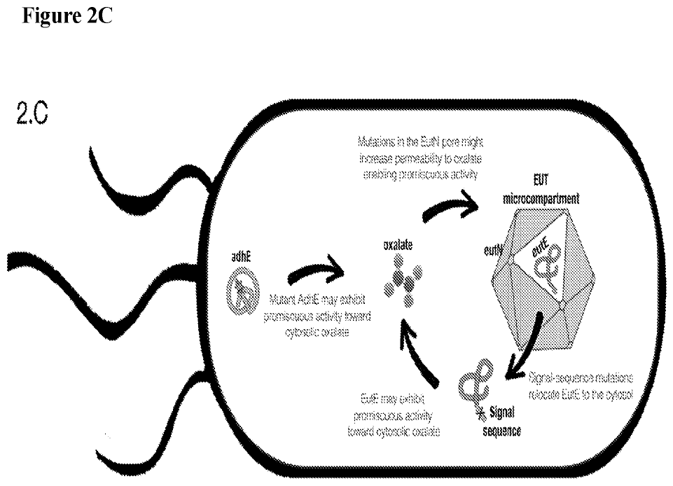

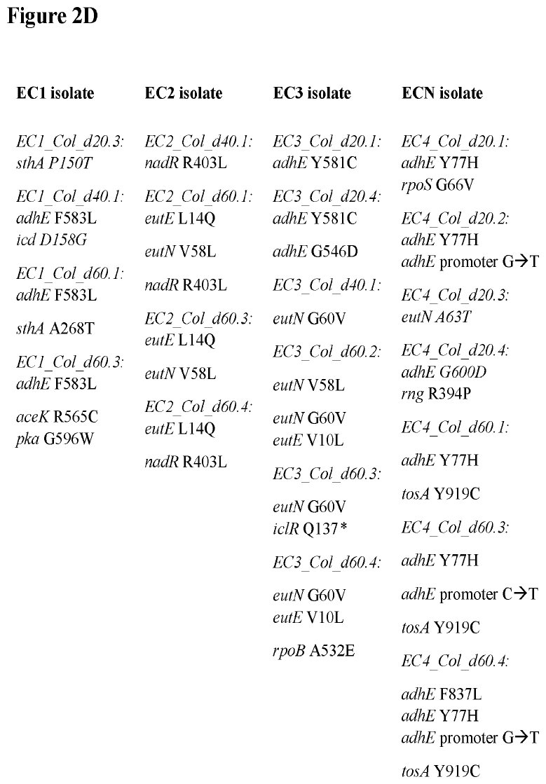

We used adaptive laboratory evolution (ALE) to select for de novo oxalate metabolism in these four gut isolates. Because these strains were isolated from the gut, and functional probiotics must also survive in the gut, we performed the lab evolution in microaerobic conditions emulating the gut. We cultured our four populations in M9 minimal medium containing 0.2% oxalate (w/v), supplemented with 0.05% glycerol (v/v), which E. coli can utilize as a source of carbon and energy. We performed 1:100 serial dilutions of each population into fresh medium every 48 hours. Every 10 dilutions, we plated samples from each evolving population on M9 agar containing 0.2% oxalate (w/v) to detect the emergence of mutants capable of utilizing oxalate as a sole source of carbon and energy. We omitted glycerol from the dilution medium for a population once samples from that population exhibited robust colonies on M9 0.2% oxalate (w/v) plates without glycerol (FIG. 1). After 60 dilutions (˜400 generations), all four populations exhibited robust growth on oxalate as a sole source of carbon and energy in microaerobic conditions. Interestingly, all of the populations evolved to utilize oxalate only microaerobically and are unable to grow on oxalate as a sole source of carbon and energy aerobically (FIG. 2A).

Repeated Mutations in Evolved Populations Suggest that De Novo Oxalate Metabolism Relies on De Novo Glyoxylate Dehydrogenase Activity.

To identify mutations involved in de novo oxalate utilization, we performed whole-genome sequencing of 10-14 colonies isolated on oxalate-only plates for each of the four evolving populations. Across populations, we identified 33 mutations, with four synonymous and three in a non-coding region. Strikingly, 15 mutations occurred in just three genes: eutE, eutN and adhE. Moreover, all three non-coding mutations observed were in the 5′-UTR of adhE (FIG. 2B).

There are several known microbial pathways for oxalate degradation, and the pathway characterized in Pseudomonas oxaliticus requires genes to which E. coli has homologs. However, E. coli lacks known homologs to a key enzyme in this pathway, glyoxylate dehydrogenase, which catalyzes the conversion of oxalyl-CoA to glyoxylate. All three of the genes in which we observe repeated mutations across populations (adhE, eutE, and eutN) code for either a CoA-acylating aldehyde dehydrogenase or a structural protein associated with a CoA-acylating aldehyde dehydrogenase: AdhE is a cytosolic bifunctional alcohol-aldehyde dehydrogenase involved in microaerobic and anaerobic ethanol production, and EutE and EutN are part of the ethanolamine-utilization (Eut) microcompartment, which is a bacterial organelle enclosed in a selectively permeable protein shell. In the Eut microcompartment, EutE is a putative acetaldehyde dehydrogenase, and EutN is a component of the shell. Notably, EutE has 56 percent sequence homology to the AdhE aldehyde dehydrogenase domain (FIG. 2C).

In FIG. 2D we report single colonies that were isolated from the four evolving populations during the lab-evolution experiment. As illustrated in FIG. 1B, during evolution each population was sampled onto agar plates that contain oxalate as the sole source of carbon and energy. Single colonies that were isolated from this process and that have shown robust ability to sustain repeated transfers on such plates were sequenced (whole genome) to detect mutations specific mutation (or mutations combinations) that confer the ability to utilize oxalate (FIG. 2D).

Key Mutations Suggest Distinct Routes to Evolved Oxalate Metabolism.

Repeated mutations in adhE, eutE, and eutN suggest that AdhE and EutE might exhibit activity toward oxalate or oxalyl-CoA, and suggest potentially convergent mechanisms for the evolved utilization of oxalate. The two mutations observed in EutE are both in EutE's N-terminal signal sequence. EutE is natively localized to the lumen of the ethanolamine-utilization microcompartment (Eut), a bacterial organelle enclosed in a proteinaceous shell that is selectively permeable to the cytosol.

The localization of Eut lumen proteins is mediated by N-terminal signal sequences of nine amino acids. Both mutations observed in EutE (L14Q in Ec2 and V10L in Ec3) are in this signal sequence and predicted to abrogate packaging of EutE into the Eut microcompartment. This is consistent with putative promiscuous activity of EutE toward cytosolic oxalate or oxalyl-CoA (FIG. 3A+B). This putative cytosolic EutE activity is also consistent with the mutations we observe in EutN. The permeability of the Eut microcompartment to the cytosol is likely determined by the central pores of its shell proteins, which include EutN. Alterations to the permeability of the Eut microcompartment mediated by EutN pore mutations may increase exposure of EutE to cytosolic oxalate or oxalyl-CoA. Strikingly, all mutations observed in EutN adjoin its central pore: V58L, which arose independently in Ec2 and Ec3; G60V in Ec3; and A63T in EcN.

To confirm whether observed mutations in the EutE signal sequence might alter localization to the cytosol, we performed Eut microcompartment encapsulation assays with wild-type and mutant EutE signal sequences fused to GFP. Since the GFP construct includes a degradation tag, fluorescence will only be observed if the signal sequence localizes the GFP construct to the microcompartment; otherwise, the GFP construct remains in the cytosol and is rapidly degraded. We compared encapsulation of the native (ancestral) EutE signal sequence (ssEutE) to the mutated EutE signal sequences ssEutE(V10L) and ssEutE(L14Q) and observed that both mutations sharply decrease localization to the microcompartment, implying that in strains with evolved EutE mutations (L14Q in EC2 and V10L in EC3) EutE is localized to the cytosol as consistent with putative non-native activity toward oxalate or oxalyl-CoA (FIG. 3C).

Attempts to purify wild-type and mutated EutEs failed due to aggregation of overexpressed proteins.

Meanwhile, the mutations we observed in AdhE are in the C-terminal alcohol-dehydrogenase (ADH) domain, and the only mutation we observe in AdhE's aldehyde-dehydrogenase (AldDH) domain, Y77H in EcN, is at the interface between the AldDH domain and the ADH domain's alcohol-binding cleft. Three of the mutations we observed in AdhE (F583L in Ec1, Y581C in Ec3, and F837L in EcN) adjoin the ADH domain's alcohol-binding cleft, and the remaining two (G546D in Ec3 and G600D in EcN) are adjacent to the alcohol-dehydrogenase active site (FIG. 4A). These mutations may alter AdhE's affinity or activity toward oxalate or oxalyl-CoA, which we sought to confirm with activity assays on purified wild-type and mutant AdhEs.

All mutants were able to utilize ethanol and acetaldehyde as substrates. Mutant AdhE proteins showed higher activities on ethanol compared to wild-type proteins. This was also seen as a drop in the Michaelis-Menten constant K_{M}. The change was more drastic for F583L and Y77H mutants. F583L mutant also showed higher activity on acetaldehyde as substrate. No activity was observed with glyoxylate, glycolate or oxalate as substrates in tested conditions. Enzymes were specific to NAD⁺ and no activity was observed on NADP⁺ as substrate. Addition of metal mix used in growth experiments did not influence substrate specificity. It is possible that secondary promiscuous activity on oxalate or glyoxylate may require additional cofactors or anaerobic conditions absent in in vitro assays.

Interestingly, the three point mutations we identified in the 5′-UTR of evolved EcN adhE are computationally inferred to increase expression of AdhE by strengthening a −10 promoter element (FIG. 4B).

### Discussion

Of known microbial pathways for oxalate utilization, the pathway characterized in Pseudomonas oxaliticus is the one for which E. coli has the largest number of necessary enzymes. However, E. coli lacks glyoxylate dehydrogenase, a key enzyme in this pathway. Our results indicate that de novo oxalate utilization pathways in E. coli rely on alterations to existing aldehyde dehydrogenases. Observed mutations in the signal sequence of EutE change the localization of the acetaldehyde dehydrogenase from the Eut microcompartment to the cytosol, exposing it to non-native substrates.

Observed mutations in EutN, a protein of the Eut microcompartment shell, might alter the permeability of the microcompartment to EutE substrates that normally cannot interact with EutE. Alternatively, observed mutations in EutN might compromise shell formation and release EutE into the cytosol. Here we show that new metabolic functions can evolve by changes in the localization of existing enzymes.

Observed mutations in AdhE, a cytosolic bifunctional ethanol and acetaldehyde dehydrogenase, cluster around the active site cleft. These mutations might also increase accessibility to non-native substrates for promiscuous reactions. Notably, though our in vitro assays of AdhE activity toward oxalate and glyoxylate were unsuccessful, we found that AdhE mutants exhibited consistently increased alcohol dehydrogenase activity, implying that AdhE mutations arising in populations evolved to utilize oxalate increase active site lability. The AdhE aldehyde dehydrogenase domain has 56 percent sequence similarity to EutE, possibly indicating convergently evolved functions in these enzymes that had previously diverged.

### REFERENCES

1. Chung, H. et al. Gut Immune Maturation Depends on Colonization with a Host-Specific Microbiota. Cell 149, 1578-1593 (2012).
2. Ainsworth, C. Therapeutic microbes to tackle disease. Nature 577, S20-S22 (2020).
3. Blacher, E. et al. Potential roles of gut microbiome and metabolites in modulating ALS in mice. Nature 572, 474-480 (2019).
4. Atarashi, K. et al. Treg induction by a rationally selected mixture of Clostridia strains from the human microbiota. Nature 500, 232-236 (2013).
5. Riglar, D. T. et al. Engineered bacteria can function in the mammalian gut long-term as live diagnostics of inflammation. Nat. Biotechnol. 35, 653-658 (2017).
6. Suez, J., Zmora, N., Segal, E. & Elinav, E. The pros, cons, and many unknowns of probiotics. Nat. Med. 25, 716-729 (2019).
7. Zmora, N. et al. Personalized Gut Mucosal Colonization Resistance to Empiric Probiotics Is Associated with Unique Host and Microbiome Features. Cell 174, 1388-1405.e21 (2018).
8. Uchimura, Y. et al. Antibodies Set Boundaries Limiting Microbial Metabolite Penetration and the Resultant Mammalian Host Response. Immunity 49, 545-559.e5 (2018).
9. Suez, J. et al. Post-Antibiotic Gut Mucosal Microbiome Reconstitution Is Impaired by Probiotics and Improved by Autologous FMT. Cell 174, 1406-1423.e16 (2018).
10. Marcobal, A. et al. A metabolomic view of how the human gut microbiota impacts the host metabolome using humanized and gnotobiotic mice. ISME J. 7, 1933-1943 (2013).
11. Zhang, C. et al. Ecological robustness of the gut microbiota in response to ingestion of transient food-borne microbes. ISME J. 10, 2235-2245 (2016).
12. Canani, R. B. et al. Probiotics for treatment of acute diarrhoea in children: randomised clinical trial of five different preparations. BMJ 335, 340 (2007).
13. Kaufman, D. W. et al. Oxalobacter formigenes May Reduce the Risk of Calcium Oxalate Kidney Stones. J. Am. Soc. Nephrol. 19, 1197-1203 (2008).
14. Mitchell, T. et al. Dietary oxalate and kidney stone formation. Am. J. Physiol. 316, F409-F413 (2019).
15. Lieske, J. C. Probiotics for prevention of urinary stones. Ann. Transl. Med. 5, 29 (2017).
16. Hoppe, B. et al. A randomised Phase I/II trial to evaluate the efficacy and safety of orally administered Oxalobacter formigenes to treat primary hyperoxaluria. Pediatr. Nephrol. 32, 781-790 (2017).
17. Milliner, D., Hoppe, B. & Groothoff, J. A randomised Phase I/III study to evaluate the efficacy and safety of orally administered Oxalobacter formigenes to treat primary hyperoxaluria. Urolithiasis 46, 313-323 (2018).
18. Hwang, I. Y. et al. Engineered probiotic Escherichia coli can eliminate and prevent Pseudomonas aeruginosa gut infection in animal models. Nat. Commun. 8, 15028 (2017).
19. Puurunen, M. K. et al. Safety and pharmacodynamics of an engineered E. coli Nissle for the treatment of phenylketonuria: a first-in-human phase 1/2a study. Nat. Metab. (2021). doi:10.1038/s42255-021-00430-7
20. Cuív, P. Ó. et al. Isolation of Genetically Tractable Most-Wanted Bacteria by Metaparental Mating. Sci. Rep. 5, 13282 (2015).
21. Zhao, S. et al. Adaptive Evolution within Gut Microbiomes of Healthy People. Cell Host Microbe 25, 656-667.e8 (2019).
22. Yona, A. H., Alm, E. J. & Gore, J. Random sequences rapidly evolve into de novo promoters. Nat. Commun. 9, 1530 (2018).
23. Blount, Z. D., Barrick, J. E., Davidson, C. J. & Lenski, R. E. Genomic analysis of a key innovation in an experimental Escherichia coli population. Nature 489, 513-8 (2012).
24. Ou, B. et al. Genetic engineering of probiotic Escherichia coli Nissle 1917 for clinical application. Appl. Microbiol. Biotechnol. 100, 8693-8699 (2016).
25. Isabella, V. M. et al. Development of a synthetic live bacterial therapeutic for the human metabolic disease phenylketonuria. Nat. Biotechnol. 36, 857-864 (2018).
26. Praveschotinunt, P. et al. Engineered E. coli Nissle 1917 for the delivery of matrix-tethered therapeutic domains to the gut. Nat. Commun. 10, 5580 (2019).
27. Keseler, I. M. et al. The EcoCyc database: reflecting new knowledge about Escherichia coli K-12. Nucleic Acids Res. 45, D543-D550 (2017).
28. Kim, G. et al. Aldehyde-alcohol dehydrogenase forms a high-order spirosome architecture critical for its activity. Nat. Commun. 10, 4527 (2019).
29. Pony, P., Rapisarda, C., Terradot, L., Marza, E. & Fronzes, R. Filamentation of the bacterial bi-functional alcohol/aldehyde dehydrogenase AdhE is essential for substrate channeling and enzymatic regulation. Nat. Commun. 11, 1426 (2020).
30. Garsin, D. A. Ethanolamine utilization in bacterial pathogens: roles and regulation. Nat. Rev. Microbiol. 8, 290-295 (2010).
31. Jakobson, C. M., Kim, E. Y., Slininger, M. F., Chien, A. & Tullman-Ercek, D. Localization of proteins to the 1,2-propanediol utilization microcompartment by non-native signal sequences is mediated by a common hydrophobic motif. J. Biol. Chem. 290, 24519-33 (2015).
32. Kerfeld, C. A., Aussignargues, C., Zarzycki, J., Cai, F. & Sutter, M. Bacterial microcompartments. Nat. Rev. Microbiol. 16, 277-290 (2018).
33. Notebaart, R. A. et al. Network-level architecture and the evolutionary potential of underground metabolism. Proc. Natl. Acad. Sci. 111, 11762-11767 (2014).
34. Solovyev, V. & Salamov, A. Automatic Annotation of Microbial Genomes and Metagenomic Sequences 3 MATERIAL AND METHODS Learning Parameters and Prediction of Protein-Coding Genes. in (2013).
35. Nichols, T. M., Kennedy, N. W. & Tullman-Ercek, D. Cargo encapsulation in bacterial microcompartments: Methods and analysis. Methods Enzymol. 617, 155-186 (2019).
36. Schneider, C. A., Rasband, W. S. & Eliceiri, K. W. NIH Image to ImageJ: 25 years of image analysis. Nat. Methods 9, 671-5 (2012).
37. Kim, E. Y. & Tullman-Ercek, D. A rapid flow cytometry assay for the relative quantification of protein encapsulation into bacterial microcompartments. Biotechnol. J. 9, 348-54 (2014).

1-69. (canceled)

70. A modified bacterium that comprises one or more mutations compared to a corresponding wild-type bacterium, wherein the modified bacterium can degrade oxalate and the corresponding wild-type bacterium cannot degrade oxalate.

71. The modified bacterium of claim 70, wherein the one or more mutations comprise mutations in one or more genes selected from the group consisting of
(a) eutE, eutN, adhE, icd, sthA, pka, aceK, nadR, iclR, rpoB, mg, tosA, and rpoS, all based on naming convention for E. coli; or homologues or orthologues thereof;
(b) eutN, adhE, sthA, and nadR; and/or
(c) eutN, adhE, and eutE.

72. The modified bacterium of claim 70, wherein the one or more mutations comprise mutations in one or more of the following combination of genes:
(a) sthA and adhE;
(b) nadR and eutE;
(c) adhE and icd;
(d) adhE and rpoS;
(e) adhE and rng;
(f) adhE and tosA;
(g) eutN and eutE;
(h) eutN and iclR;
(i) nadR, eutE, and eutN;
(j) adhE, aceK, and pka; and/or
(k) eutN, eutE, and rpoB.

73. The modified bacterium of claim 70 wherein the one or more mutations comprise one or more mutations in a signal sequence-encoding portion of the eutE gene, wherein
(a) the one or more mutations comprise a deletion of all or a portion of the eutE gene encoding the signal sequence; or
(b) the one or more mutations in the signal sequence-encoding portion of the eutE gene result in the eutE gene encoding a EutE protein comprising mutations at one or both of residues 10 and 14, based on residue numbering for E. coli EutE protein sequence; optionally wherein the mutations at one or both of residues 10 and 14 comprise one or both of mutations V10L and/or L14Q mutations, numbering and residues based on the E. coli EutE protein sequence.

74. The modified bacterium of claim 70 wherein the one or more mutations comprise one or more mutations in the eutN gene encoding pore-facing amino acid residues, wherein:
(a) the one or more mutations comprise one or more mutations in the eutN gene encoding pore-facing amino acid residues 58, 60, and 63, numbering and residues based on the E. coli EutN protein sequence; or
(b) the one or more mutations result in the eutN gene encoding a EutN protein comprising one or more mutation selected from V58L, G60V, and A63T, numbering and residues based on the E. coli EutN protein sequence.

75. The modified bacterium of claim 70, wherein the one or more mutations comprise one or more mutations in the nadR gene; wherein the one or more mutations result in the nadR gene encoding a NadR protein comprising a mutation at residue 403, numbering and residues based on the E. coli NadR protein sequence, optionally wherein the mutation at residue 403 comprises a R403L mutation.

76. The modified bacterium of claim 70, wherein the one or more mutations comprise one or more mutations in the sthA gene, wherein the one or more mutations result in the sthA gene encoding a SthA protein comprising a mutation at one or both of residues 150 and 268, numbering and residues based on the E. coli NadR protein sequence; optionally wherein
(a) the mutation at one or both of residues 150 and 268 comprises P150T and/or A268T mutations; or
(b) the one or more mutations result in the sthA gene encoding a SthA protein comprising a P150T mutation, numbering and residues based on the E. coli NadR protein sequence.

77. The modified bacterium of claim 70, wherein the modified bacterium comprises one or more mutations that result in genes encoding one or more sets of mutated proteins selected from the group consisting of:
(a) SthA P150T;
(b) AdhE F583L and Icd D158G;
(c) AdhE F583L and SthA A268T;
(d) AdhE F583L, AceK R565C, and Pka G596W;
(e) NadR R403L;
(f) EutE L14Q, EutN V58L, and NadR R403L:
(g) EutE L14Q and EutN V58L;
(h) EutE L14Q and NadR R403L;
(i) AdhE Y581C;
(j) AdhE Y581C and AdhE G546D;
(k) EutN G60V;
(l) EutN V58L, EutN G60V, EutE V10L;
(m) EutN G60V and a knockout or truncation mutation of IclR, including but not limited to Q137*, wherein the asterisk represents a truncation at residue 137
(n) EutN G60V, EutE V10L, and RpoB A532E;
(o) AdhE Y77H and RpoS G66V;
(p) AdhE Y77H;
(q) EutN A63 T;
(r) AdhE G600D and Rng R394P;
(s) AdhE Y77H and TosA Y919C; and
(t) AdhE F837L, AdhE Y77H, and TosA Y919C,
numbering and residues based on the E. coli protein sequences.

78. The modified bacterium of claim 70, comprising
(a) a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 1, and including 1, 2, 3, 4, or all 5 residues selected from 77H, 6546D, 581C, 600D, and/or F837L;
(b) a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 1, and including 2, 3, 4, 5, or all 6 residues selected from 77H, 6546D, 581C, F583L, 600D, and/or F837L;
(c) a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 2, and including one both residues selected from 10L and 14Q
(d) a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 3, and including 1, 2, or all 3 residues selected from 58L, 60V, and 63T;
(e) a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 4, and including residue 403L; and/or
(f) a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of SEQ ID NO: 5 and including residue 150T and/or A268T.

79. The modified bacterium of claim 70, comprising a gene encoding a protein comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of one or more of the following:
(a) SEQ ID NO: 6 and including residue D158G;
(b) SEQ ID NO:7 and including residue R565C;
(c) SEQ ID NO:8 and including residue Q137*, wherein the asterisk indicates a truncation of the protein at residue 137;
(d) SEQ ID NO:9 and including residue A532E;
(e) SEQ ID NO:10 and including residue G66V;
(f) SEQ ID NO:11 and including residue R394P;
(g) SEQ ID NO:12 and including residue Y919C; and/or
(h) SEQ ID NO:13 and including residue G596W.

80. The modified bacterium of claim 70, further comprising one or more mutations in the adhE gene promoter sequence; optionally wherein the one or more mutations in the adhE gene promoter sequence comprise one or more A to C mutations in the region 95 to 150 nucleotides upstream of the adhE coding sequence start codon, for example, at nucleotides 104, 114, and/or 128 residues upstream of the start codon.

81. The modified bacterium of claim 70, wherein the one or more mutated gene is operatively linked to a recombinant regulatory sequence comprising a heterologous promoter; optionally wherein the promoter is an inducible promoter, optionally wherein the inducible promoter is inducible by exogenous environmental conditions found in the mammalian gut or by low-oxygen or anaerobic conditions; optionally wherein the inducible promoter is selected from the group consisting of an FNR-responsive promoter, an ANR-responsive promoter, and a DNR-responsive promoter.

82. The modified bacterium of claim 70, wherein the bacterium is a member of a genus selected from the group consisting of Bacteroides, Bifidobacterium, Clostridium, Escherichia, Streptococcus, Lactobacillus and Lactococcus.

83. A bacterial population, comprising a plurality of the modified bacterium of claim 70.

84. An isolated protein, comprising an amino acid sequence at least 40%, 45%, 50%, 55%, 60%, 65%, 70%, 75%, 80%, 85%, 90%, 91%, 92%, 93%, 94%, 95%, 96%, 97%, 95%, 99%, or 100% identical to the amino acid sequence of one of the following:
(a) SEQ ID NO: 1, and including 1, 2, 3, 4, or all 5 residues selected from 77H, 546D, 581C, 600D, and/or F837L;
(b) SEQ ID NO: 1, and including 2, 3, 4, 5, or all 6 residues selected from 77H, 546D, 581C, F583L, 600D, and/or F837L;
(c) SEQ ID NO: 2, and including one or both residues selected from 10L and 14Q;
(d) SEQ ID NO: 3, and including one or more residues selected from 58L, 60V, and 63T;
(e) SEQ ID NO: 4, and including residue 403L;
(f) SEQ ID NO: 5 and including residue 150T and/or A268T;
(g) SEQ ID NO: 6 and including residue D158G;
(h) SEQ ID NO:7 and including residue R565C;
(i) SEQ ID NO:8 and including residue Q137*, wherein the asterisk represents a truncation of the protein at residue 137;
(j) SEQ ID NO:9 and including residue A532E;
(k) SEQ ID NO:10 and including residue G66V;
(l) SEQ ID NO:11 and including residue R394P;
(m) SEQ ID NO:12 and including residue Y919C; and
(n) SEQ ID NO:13 and including residue G596W.

85. A nucleic acid encoding the polypeptide of claim 84.

86. A composition comprising the modified bacterium of claim 70.

87. A method for reducing oxalate, treating or limiting development of a disorder in which oxalate is detrimental in a subject, treating or limiting development of hyperoxaluria, or treating or limiting development of kidney stones, comprising administering to a subject in need thereof an amount effective to reduce oxalate of the modified bacterium of claim 70.

88. A method of producing bacteria that can degrade oxalate, comprising
(a) culturing a bacterial population in an anaerobic chamber in a medium comprising oxalate supplemented with glycerol;
(b) performing 1:100 dilutions of the population into fresh medium every 48 hours; and
(c) every 10 dilutions, plating samples of the population onto agar comprising oxalate to detect mutants capable of degrading oxalate.


SEQUENCE LISTING (20 sequences)

SEQ ID NO: 1 - protein, 891 aa, Artificial Sequence
  Synthetic
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 2 - protein, 467 aa, Artificial Sequence
  Synthetic
MNQQDIEQVVKAVLLKMQSSDTPSAAVHEMGVFASLDDAVAAAKVAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAGKIAAAIGLKVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 3 - protein, 95 aa, Artificial Sequence
  Synthetic
MKLAVVTGQIVCTVRHHGLAHDKLLMVEMIDPQGNPDGQCAVAIDNIGAGTGEWVLLVSGSSARQAHKSETSPVDLCVIGIVDEVVSGGQVIFHK

SEQ ID NO: 4 - protein, 410 aa, Artificial Sequence
  Synthetic
MSSFDYLKTAIKQQGCTLQQVADASGMTKGYLSQLLNAKIKSPSAQKLEALHRFLGLEFPRQKKTIGVVFGKFYPLHTGHIYLIQRACSQVDELHIIMGFDDTRDRALFEDSAMSQQPTVPDRLRWLLQTFKYQKNIRIHAFNEEGMEPYPHGWDVWSNGIKKFMAEKGIQPDLIYTSEEADAPQYMEHLGIETVLVDPKRTFMSISGAQIRENPFRYWEYIPTEVKPFFVRTVAILGGESSGKSTLVNKLANIFNTTSAWEYGRDYVFSHLGGDEIALQYSDYDKIALGHAQYIDFAVKYANKVAFIDTDFVTTQAFCKKYEGREHPFVQALIDEYRFDLVILLENNTPWVADGLRSLGSSVDRKEFQNLLVEMLEENNIEFVRVEEEDYDSRFLRCVELVREMMGEQR

SEQ ID NO: 5 - protein, 466 aa, Artificial Sequence
  Synthetic
MPHSYDYDAIVIGSGPGGEGAAMGLVKQGARVAVIERYQNVGGGCTHWGTIPSKALRHAVSRIIEFNQNPLYSDHSRLLRSSFADILNHADNVINQQTRMRQGFYERNHCEILQGNARFVDEHTLALDCPDGSVETLTAEKFVIACGSRPYHPTDVDFTHPRIYDSDSILSMHHEPRHVLIYGAGVIGCEYASIFRGMDVKVDLINTRDRLLAFLDQEMSDSLSYHFWNSGVVIRHNEEYEKIEGCDDGVIMHLKSGKKLKADCLLYANGRTGNTDSLALQNIGLETDSRGQLKVNSMYQTAQPHVYAVGDVIGYPSLASAAYDQGRIAAQALVKGEATAHLIEDIPTGIYTIPEISSVGKTEQQLTAMKVPYEVGRAQFKHLARAQIVGMNVGTLKILFHRETKEILGIHCFGERAAEIIHIGQAIMEQKGGGNTIEYFVNTTFNYPTMAEAYRVAALNGLNRLF

SEQ ID NO: 6 - protein, 416 aa, Artificial Sequence
  Synthetic
MESKVVVPAQGKKITLQNGKLNVPENPIIPYIEGDGIGVDVTPAMLKVVDAAVEKAYKGERKISWMEIYTGEKSTQVYGQDVWLPAETLDLIREYRVAIKGPLTTPVGGGIRSLNVALRQELDLYICLRPVRYYQGTPSPVKHPELTDMVIFRENSEDIYAGIEWKADSADAEKVIKFLREEMGVKKIRFPEHCGIGIKPCSEEGTKRLVRAAIEYAIANDRDSVTLVHKGNIMKFTEGAFKDWGYQLAREEFGGELIDGGPWLKVKNPNTGKEIVIKDVIADAFLQQILLRPAEYDVIACMNLNGDYISDALAAQVGGIGIAPGANIGDECALFEATHGTAPKYAGQDKVNPGSIILSAEMMLRHMGWTEAADLIVKGMEGAINAKTVTYDFERLMDGAKLLKCSEFGDAIIENM

SEQ ID NO: 7 - protein, 578 aa, Artificial Sequence
  Synthetic
MPRGLELLIAQTILQGFDAQYGRFLEVTSGAQQRFEQADWHAVQQAMKNRIHLYDHHVGLVVEQLRCITNGQSTDAAFLLRVKEHYTRLLPDYPRFEIAESFFNSVYCRLFDHRSLTPERLFIFSSQPERRFRTIPRPLAKDFHPDHGWESLLMRVISDLPLRLRWQNKSRDIHYIIRHLTETLGTDNLAESHLQVANELFYRNKAAWLVGKLITPSGTLPFLLPIHQTDDGELFIDTCLTTTAEASIVFGFARSYFMVYAPLPAALVEWLREILPGKTTAELYMAIGCQKHAKTESYREYLVYLQGCNEQFIEAPGIRGMVMLVFTLPGFDRVFKVIKDRFAPQKEMSAAHVRACYQLVKEHDRVGRMADTQEFENFVLEKRHISPALMELLLQEAAEKITDLGEQIVIRHLYIERRMVPLNIWLEQVEGQQLRDAIEEYGNAIRQLAAANIFPGDMLFKNFGVTRHGRVVFYDYDEICYMTEVNFRDIPPPRYPEDELASEPWYSVSPGDVFPEEFRHWLCADPRIGPLFEEMHADLFRADYWRALQNRIREGHVEDVYAYRRRQRFSVRYGEMLF

SEQ ID NO: 8 - protein, 274 aa, Artificial Sequence
  Synthetic
MVAPIPAKRGRKPAVATAPATGQVQSLTRGLKLLEWIAESNGSVALTELAQQAGLPNSTTHRLLTTMQQQGFVRQVGELGHWAIGAHAFMVGSSFLQSRNLLAIVHPILRNLMEESGETVNMAVLDQSDHEAIIIDQVQCTHLMRMSAPIGGKLPMHASGAGKAFLAQLSEEQVTKLLHRKGLHAYTHATLVSPVHLKEDLAQTRKRGYSFDDEEHALGLRCLAACIFDEHREPFAAISISGPISRITDDRVTEFGAMVIKAAKEVTLAYGGMR

SEQ ID NO: 9 - protein, 1342 aa, Artificial Sequence [sequence omitted]

SEQ ID NO: 10 - protein, 330 aa, Artificial Sequence
  Synthetic
MSQNTLKVHDLNEDAEFDENGVEVFDEKALVEQEPSDNDLAEEELLSQGATQRVLDATQLYLGEIGYSPLLTAEEEVYFARRALRGDVASRRRMIESNLRLVVKIARRYGNRGLALLDLIEEGNLGLIRAVEKFDPERGFRFSTYATWWIRQTIERAIMNQTRTIRLPIHIVKELNVYLRTARELSHKLDHEPSAEEIAEQLDKPVDDVSRMLRLNERITSVDTPLGGDSEKALLDILADEKENGPEDTTQDDDMKQSIVKWLFELNAKQREVLARRFGLLGYEAATLEDVGREIGLTRERVRQIQVEGLRRLREILQTQGLNIEALFRE

SEQ ID NO: 11 - protein, 489 aa, Artificial Sequence
  Synthetic
MTAELLVNVTPSETRVAYIDGGILQEIHIEREARRGIVGNIYKGRVSRVLPGMQAAFVDIGLDKAAFLHASDIMPHTECVAGEEQKQFTVRDISELVRQGQDLMVQVVKDPLGTKGARLTTDITLPSRYLVFMPGASHVGVSQRIESESERERLKKVVAEYCDEQGGFIIRTAAEGVGEAELASDAAYLKRVWTKVMERKKRPQTRYQLYGELALAQRVLRDFADAELDRIRVDSRLTYEALLEFTSEYIPEMTSKLEHYTGRQPIFDLFDVENEIQRALERKVELKSGGYLIIDQTEAMTTVDINTGAFVGHRNLDDTIFNTNIEATQAIARQLRLRNLGGIIIIDFIDMNNEDHRRRVLHSLEQALSKDRVKTSVNGFSALGLVEMTRKRTRESIEHVLCNECPTCHGRGTVKTVETVCYEIMREIVRVHHAYDSDRFLVYASPAVAEALKGEESHSLAEVEIFVGKQVKVQIEPLYNQEQFDVVMM

SEQ ID NO: 12 - protein, 940 aa, Artificial Sequence
  Synthetic
MKMIFTGKVSGEKTVLTVGGRHTVKAQPGEQYGLIDEVTGLVPDGVEADRSGDDLILRKKEEDTEVRIEGFWEECQPGETQCTAVFNIVGEDGQVTEAVLTQDGPVLDDITAGQSGTLSDDDRGGFIWLGGLAFGGGLAAMALAAGGGGSKHRHENDDSDTTAPSSPALKAEDDGSVSVELPGDANKGDTVDVTFEDEKGGKHTVTLEKGDNGWTSSDPTLIPDSTGDKATIPADNVKDNSEVTGVAKDPSGNESDPSTVTSKTDVLPTVSISVETTSTDVNGDGFTGIASVNGTVMDVPATIEDKDDSTGLVYTVSLNHVTTTDVTVTVTLGSGAGHSDAADYSDIGGAQHNGKIGLHGDTGKVTYDGATTVTIVIPAGSKSVSFIVDPTLEANQDAFNAEGMEKVVATITGTSDNVTAATDIVDNAGASATGVIYDGNAISLRNLDGDFTLKYSLSSSIAEKGDFGYTIGANSGENDPMVTTDYNDTVYVGYYQSGKETTSYSNVANSQDNGPDGTKTDGNQSITTVDLGAGDDLMVIRGNMLANTRVYTGEGNDTFTMDGMNTALRVMYAGSYIFTESGDDIVTIKRTGVTNAGQIYLGSGSDTFIQGDATDNNDTTLSGLLDLGSGTKDISNMPKEYLSVYQDGSNLSLGNDNNIDTATDVNTVTIYGSVSGEILGGYGSDNITVTKNLTGNISVGDNADTLTAGWIYGGSTVSMGDGNDTVTITDGAYNTTISLGAGDDVFDATSGVMGDSAYATVVNGEDGNDTFKLGTIAKNLTIDAGAGDDIVVLTKDYDSTSSGNQGYINGGEGSDTLVLTGTISVNLAAGKNEGIAGFEKVDMTVGSDLKADNAAQLVKLTASDVLGINDNSTIYISGDANDKVDLGADGAGSLGTFTATATTVKATALDGIEHTYTLYSSVSGANVYIDNNIIDANGVI

SEQ ID NO: 13 - protein, 886 aa, Artificial Sequence
  Synthetic
MSQRGLEALLRPKSIAVIGASMKPNRAGYLMMRNLLAGGFNGPVLPVTPAWKAVLGVLAWPDIASLPFTPDLAVLCTNASRNLALLEELGEKGCKTCIILSAPASQHEDLRACALRHNMRLLGPNSLGLLAPWQGLNASFSPVPIKRGKLAFISQSAAVSNTILDWAQQRKMGFSYFIALGDSLDIDVDELLDYLARDSKTSAILLYLEQLSDARRFVSAARSASRNKPILVIKSGRSPAAQRLLNTTAGMDPAWDAAIQRAGLLRVQDTHELFSAVETLSHMRPLRGDRLMIISNGAAPAALALDALWSRNGKLATLSEETCQKLRDALPEHVAISNPLDLRDDASSEHYIKTLDILLHSQDFDALMVIHSPSAAAPATESAQVLIEAVKHHPRSKYVSLLTNWCGEHSSQEARRLFSEAGLPTYRTPEGTITAFMHMVEYRRNQKQLRETPALPSNLTSNTAEAHLLLQQAIAEGATSLDTHEVQPILQAYGMNTLPTWIASDSTEAVHIAEQIGYPVALKLRSPDIPHKSEVQGVMLYLRTANEVQQAANAIFDRVKMAWPQARVHGLLVQSMANRAGAQELRVVVEHDPVFGPLIMLGEGGVEWRPEDQAVVALPPLNMNLARYLVIQGIKSKKIRARSALRPLDVAGLSQLLVQVSNLIVDCPEIQRLDIHPLLASGSEFTALDVTLDISPFEGDNESRLAVRPYPHQLEEWVELKNGERCLFRPILPEDEPQLQQFISRVTKEDLYYRYFSEINEFTHEDLANMTQIDYDREMAFVAVRRIDQTEEILGVTRAISDPDNIDAEFAVLVRSDLKGLGLGRRLMEKLITYTRDHGLQRLNGITMPNNRGMVALARKLGFNVDIQLEEGIVGLTLNLAQREES

SEQ ID NO: 14 - protein, 20 aa, Artificial Sequence
  Synthetic
MNQQDIEQVLKAVQLKMQSS

SEQ ID NO: 15 - protein, 20 aa, Artificial Sequence
  Synthetic
MEINEKLLRQIIEDVLRDMK

SEQ ID NO: 16 - protein, 18 aa, Artificial Sequence
  Synthetic
MNTSELETLIRTILSEQL

SEQ ID NO: 17 - protein, 18 aa, Artificial Sequence
  Synthetic
MNQQDIEQVVKAVLLKMK

SEQ ID NO: 18 - protein, 18 aa, Artificial Sequence
  Synthetic
MNQQDIEQVLKAVLLKMK

SEQ ID NO: 19 - protein, 18 aa, Artificial Sequence
  Synthetic
MNQQDIEQVVKAVQLKMK

SEQ ID NO: 20 - DNA, 61 bp, Artificial Sequence
  Synthetic
tagagataataactctaatgtttaaaatcttttagtaaataacagtgagtatgagcgcgag